SEQUENCE LISTING (1 sequence)

SEQ ID NO: 1 - protein, 431 aa, Candida antarctica
AALPNPYDDPFYTTPSNIGTFAKGQVIQSRKVPTDIGNANNAASFQLQYRTTNTQNEAVADVATVWIPAKPASPPKIFSYQVYEDATALDCAPSYSYLTGLDQPNKVTAVLDTPIIIGWALQQGYYVVSSDHEGFKAAFIAGYEEGMAILDGIRALKNYQNLPSDSKVALEGYSGGAHATVWATSLAESYAPELNIVGASHGGTPVSAKDTFTFLNGGPFAGFALAGVSGLSLAHPDMESFIEARLNAKGQRTLKQIRGRGFCLPQVVLTYPFLNVFSLVNDTNLLNEAPIASILKQETVVQAEASYTVSVPKFPRFIWHAIPDEIVPYQPAATYVKEQCAKGANINFSPYPIAEHLTAEIFGLVPSLWFIKQAFDGTTPKVICGTPIPAIAGITTPSADQVLGSDLANQLRSLDGKQSAFGKPFGPITPP

(19) United States
(12) Patent Application Publication
(10) Pub. No.: US 2010/0003708 A1
(43) Pub. Date: Jan. 7, 2010

(54) LIPOLYTIC ENZYME VARIANTS

(75) Inventors: Allan Svendsen, Hoersholm (DK); Jesper Vind, Vaerloese (DK); Shamkant Anant Patkar, Lyngby (DK); Kim Borch, Birkeroed (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/443,578

(22) PCT Filed: Oct. 2, 2007
(86) PCT No.: PCT/EP2007/060472

§ 371 Date: Apr. 8, 2009

Related U.S. Application Data
(60) Provisional application No. 60/848,953, filed on Oct. 3, 2006.

(30) Foreign Application Priority Data
Oct. 3, 2006 (EP) 06121676.8

(51) Int. Cl. C12Q 1/34; C12N 9/14

(52) U.S. Cl. 435/18; 435/195

(57) ABSTRACT

Lipolytic enzyme variants with increased specificity for short-chain fatty acids can be designed on the basis of a three-dimensional model of a lipolytic enzyme such as C. antarctica lipase A with a substrate analogue such as a fatty acid. An amino acid residue is selected within 10 Å of the carbon atom corresponding to the desired chain-length specificity, and the selected residue is substituted with a larger residue, or an amino acid insertion is made adjacent to the selected residue.

### FIELD OF THE INVENTION

The present invention relates to a polypeptide with lipolytic enzyme activity and to a method of preparing it.

### BACKGROUND OF THE INVENTION

Lipolytic enzymes are polypeptides with hydrolytic activity towards fatty acid ester bonds in a variety of substrates. In some applications, it is of interest to use an enzyme with a high selectivity for short-chain fatty acyl bonds.

WO8802775A1 describes Candida antarctica lipase A and its substrate specificity. WO0032758A1 (particularly Examples 10-12) discloses lipase variants with increased specificity for short-chain fatty acids. WO9401541A1 discloses variants of C. antarctica lipase A.

### SUMMARY OF THE INVENTION

The inventors have found that lipolytic enzyme variants with increased specificity for short-chain fatty acids can be designed on the basis of a three-dimensional model of a lipolytic enzyme such as C. antarctica lipase A with a substrate analogue such as a fatty acid. An amino acid residue is selected within 10 Å of the carbon atom corresponding to the desired chain-length specificity, and the selected residue is substituted with a larger residue, or an amino acid insertion is made adjacent to the selected residue.

Accordingly, the invention provides a method of preparing a polypeptide, comprising

a) providing a three dimensional model of a parent polypeptide having lipolytic enzyme activity and at least 80% identity to SEQ ID NO: 1 and a substrate analogue comprising a straight-chain fatty acyl group,

b) selecting a chain length (n) and identifying the corresponding carbon atom in the fatty acyl group,

c) selecting an amino acid residue in SEQ ID NO: 1 which has a non-hydrogen atom within 10 Å of the selected carbon atom in the model,

d) providing an altered amino acid sequence which is at least 80% identical to SEQ ID NO: 1, and wherein the difference from SEQ ID NO: 1 comprises substitution of the selected residue or insertion of at least one residue adjacent to the selected residue,

e) preparing an altered polypeptide having the altered amino acid sequence,

f) determining the hydrolytic activity of the altered polypeptide on fatty acyl ester bonds in two substrates having fatty acyl groups with different length, and

g) selecting an altered polypeptide which has an altered chain-length specificity compared to the polypeptide of SEQ ID NO: 1.

The invention also provides a polypeptide which:

has lipolytic enzyme activity, and

has an amino acid sequence which has at least 80% identity to SEQ ID NO: 1 and has a different residue at a position or has an insertion adjacent to a position corresponding to any of residues 82-87, 108, 132-133, 138, 140-142, 145, 172-179, 182, 202-216, 220-232, 235, 238, 241-242, 257,264, 267-268, 275-277, 280, 282-288, 290-296, 298-299, 304, 320, 324-328, 356-357, 360 and 420-421 of SEQ ID NO: 1.

### BRIEF DESCRIPTION OF DRAWINGS

FIG. 1 gives the coordinates for a 3D model of C. antarctica lipase A (SEQ ID NO: 1) with myristic acid as a substrate analogue.

### DETAILED DESCRIPTION OF THE INVENTION

### Parent Polypeptide

The invention uses a parent polypeptide with lipolytic enzyme activity. It may be Candida antarctica lipase A having the amino acid sequence shown in SEQ ID NO: 1, or it may be the lipase from Pseudozyma sp. described in WO2005040334 or another polypeptide whose sequence is at least 80% identical to one of those.

### Three-Dimensional Model

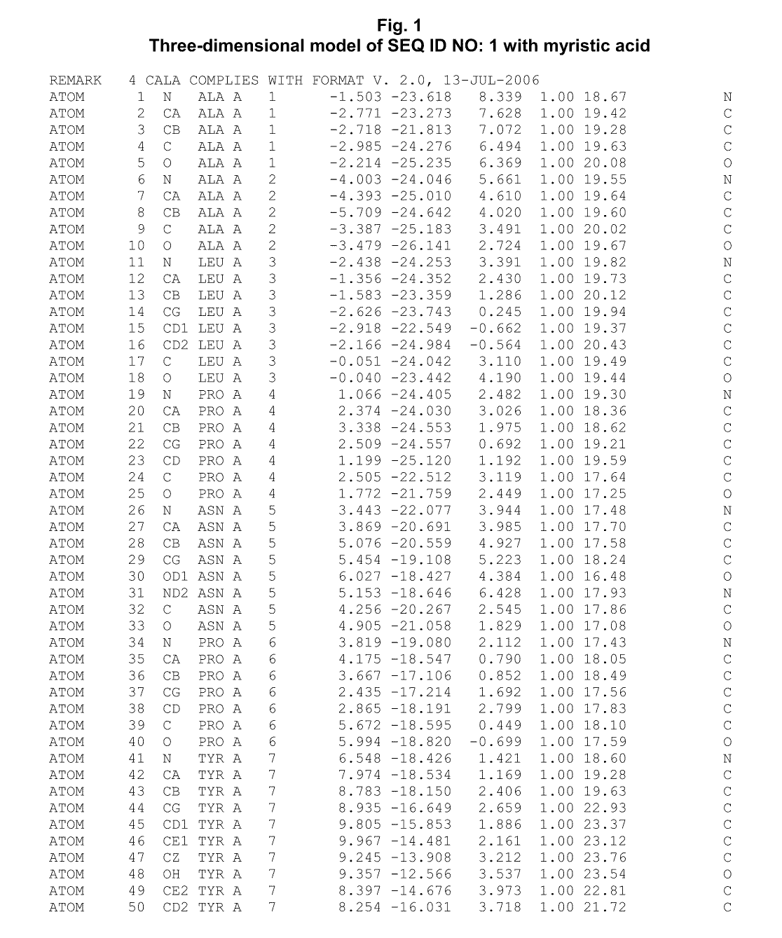

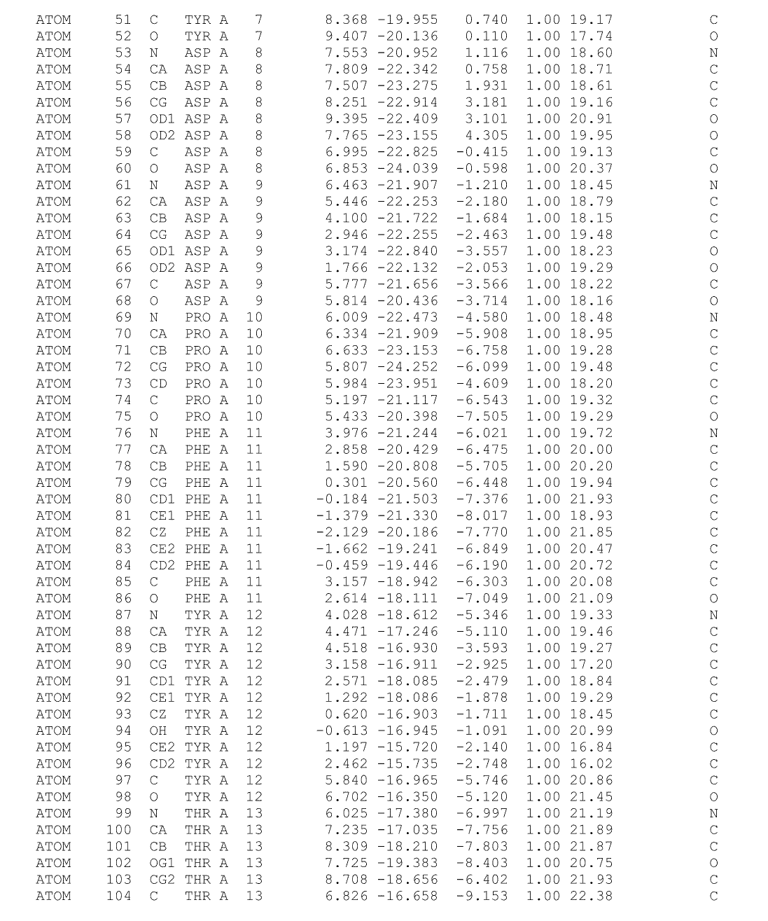

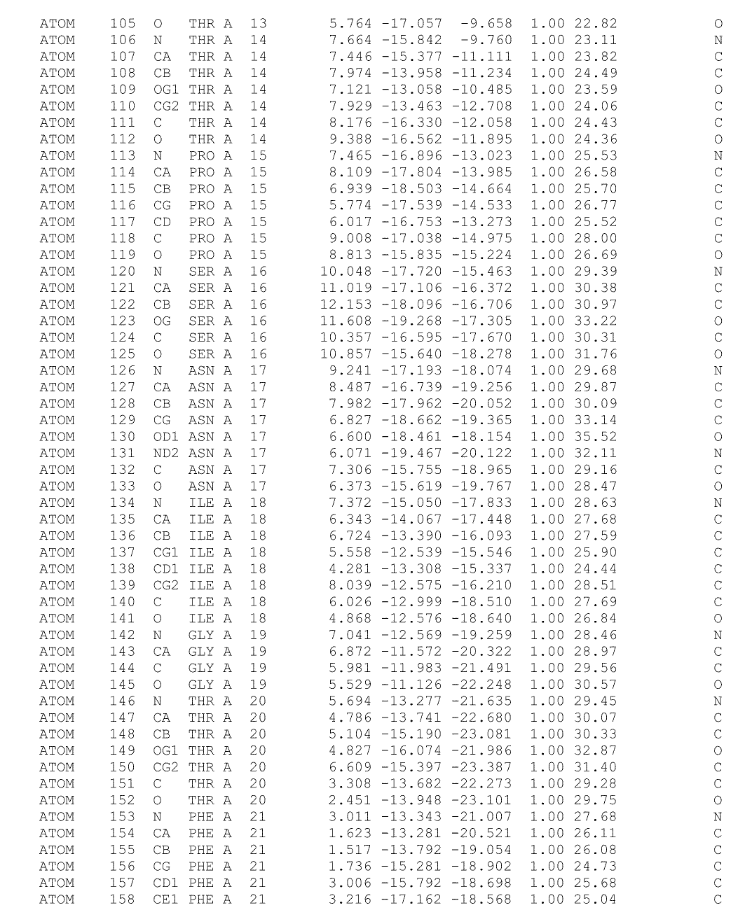

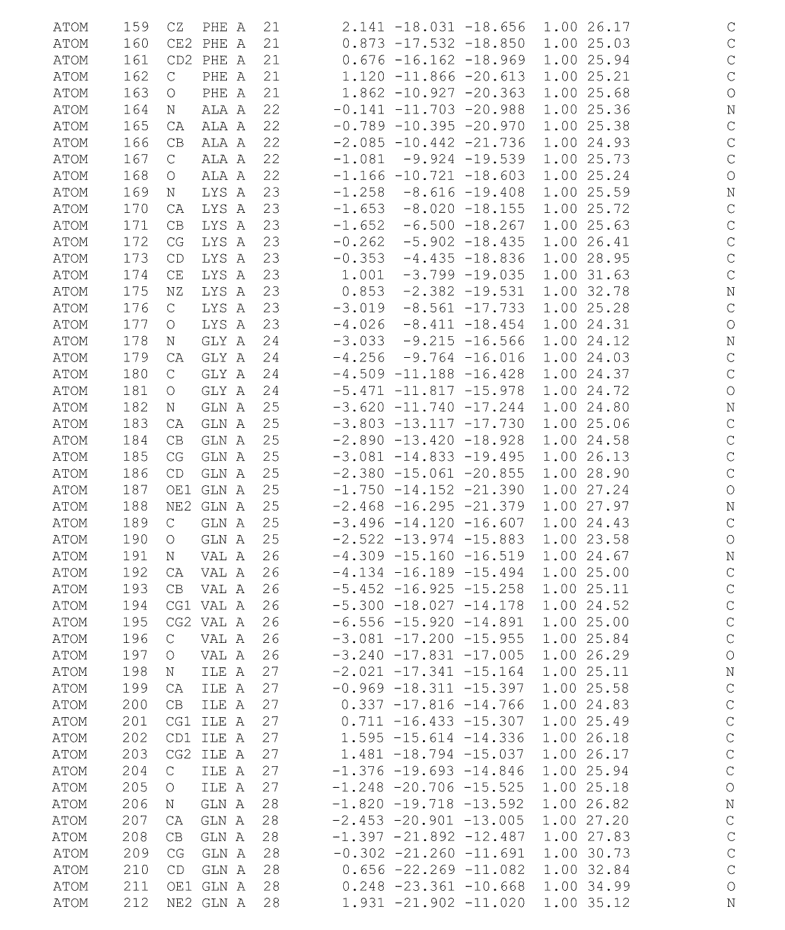

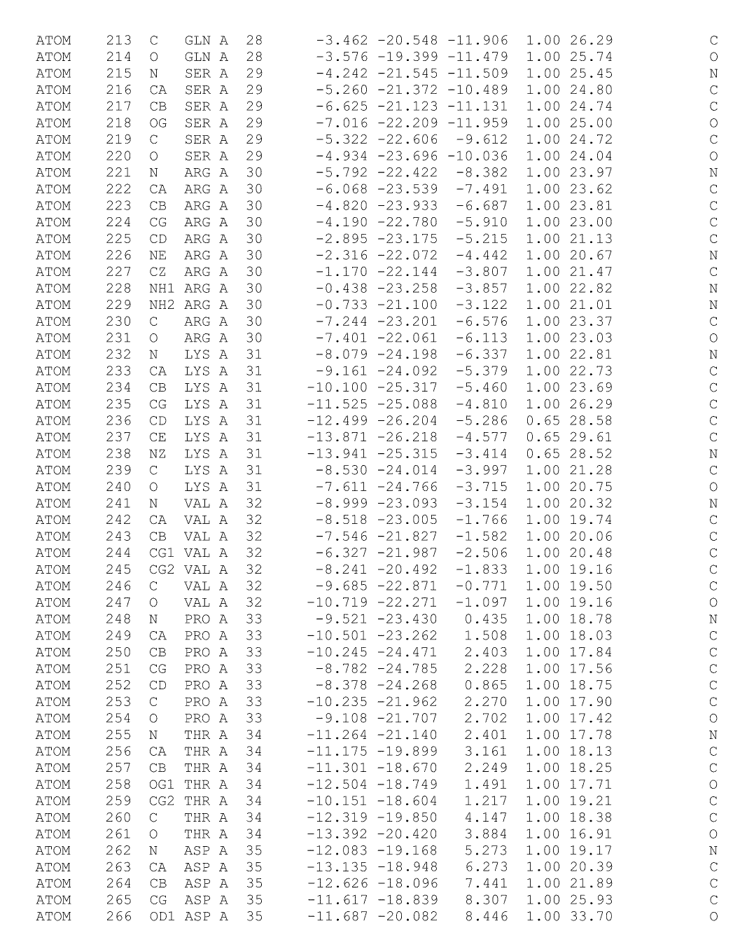

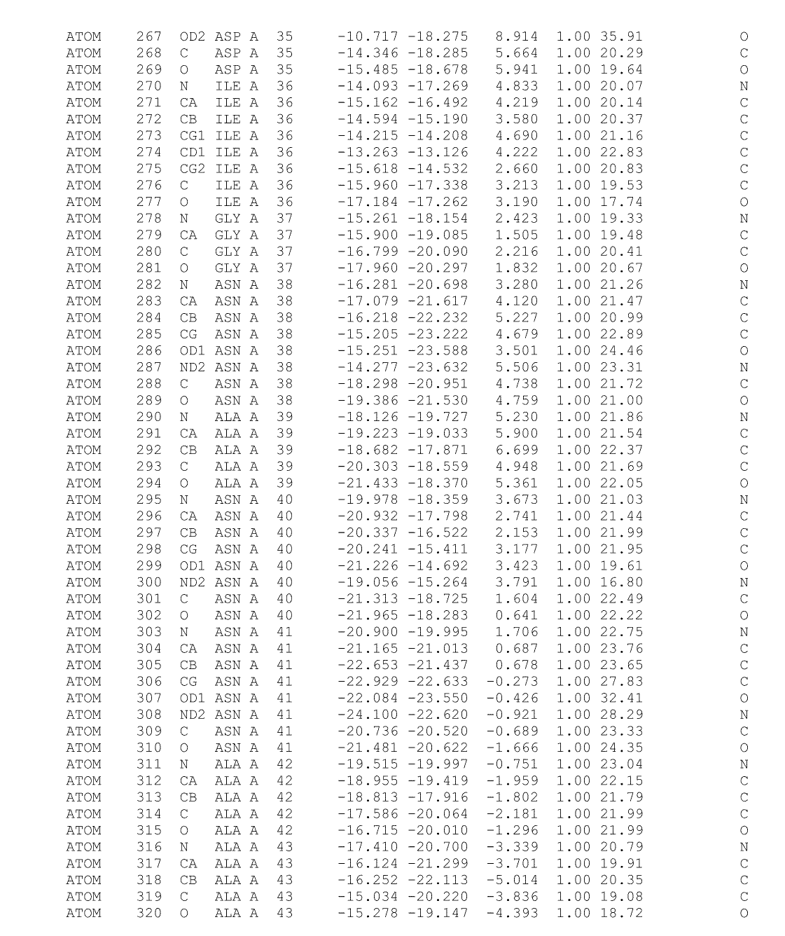

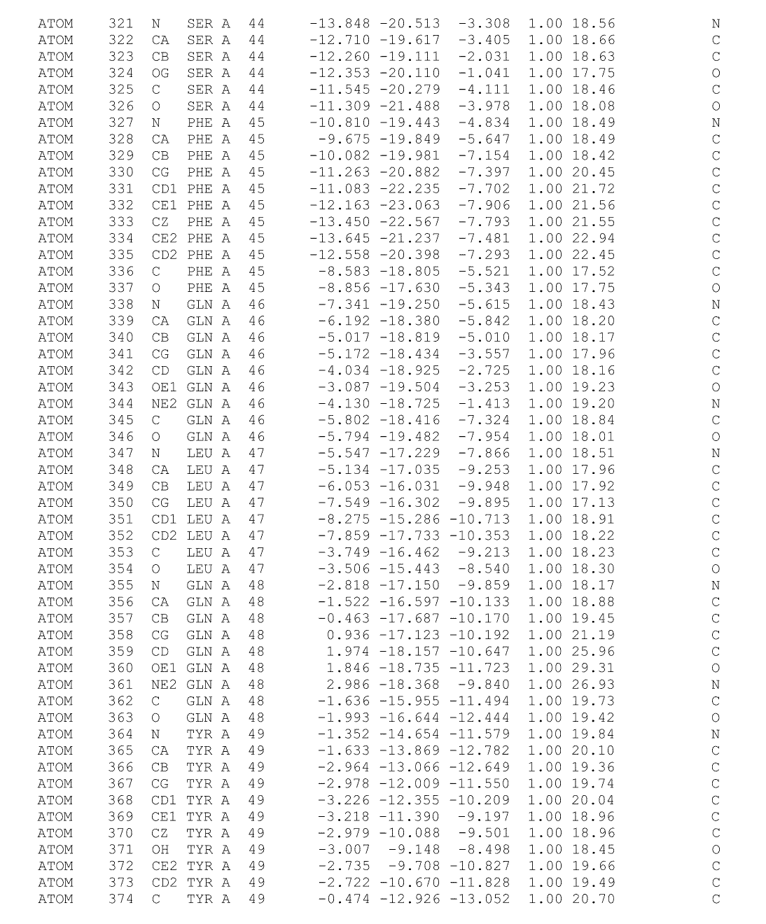

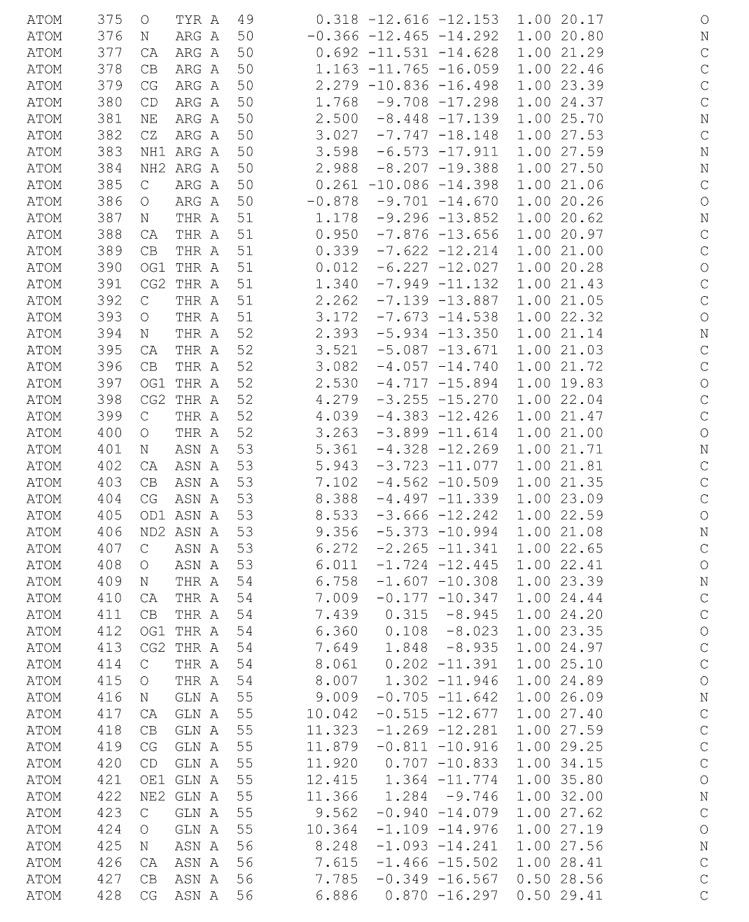

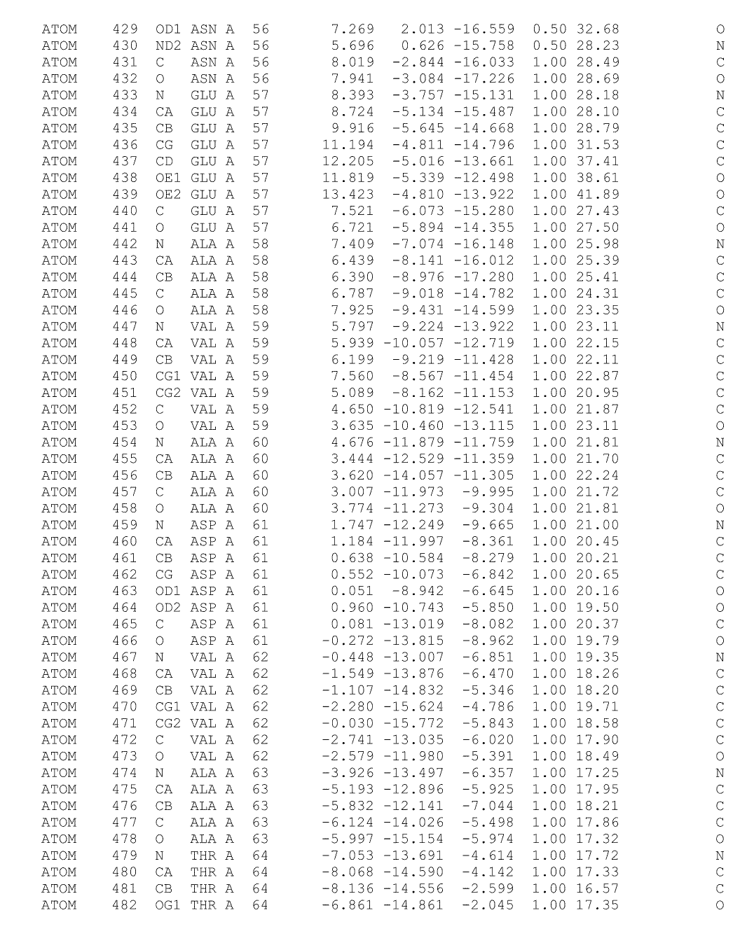

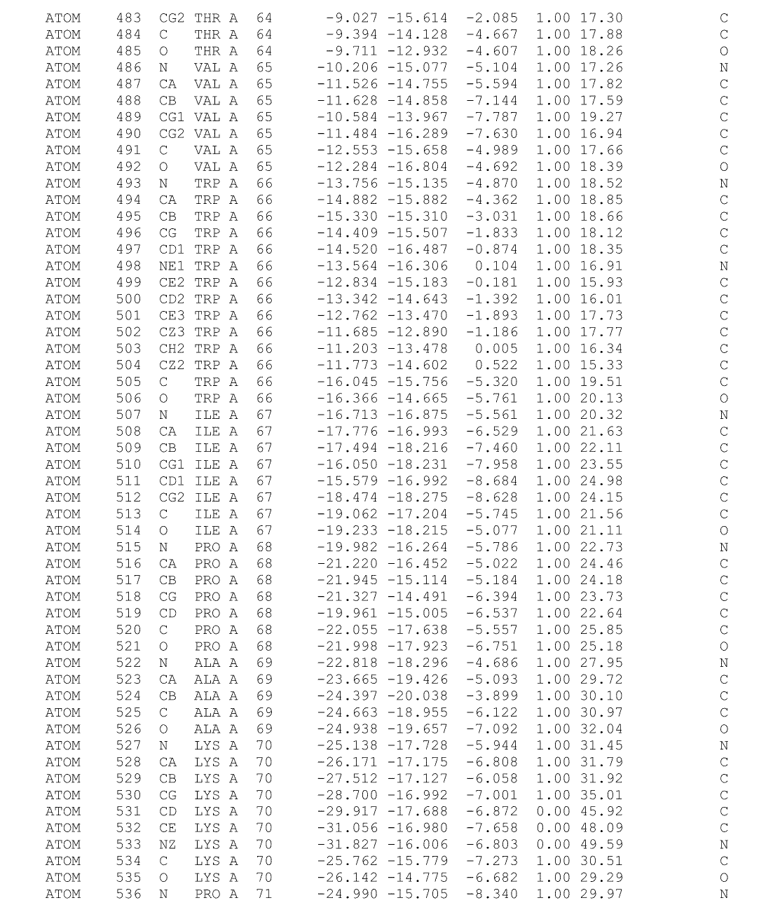

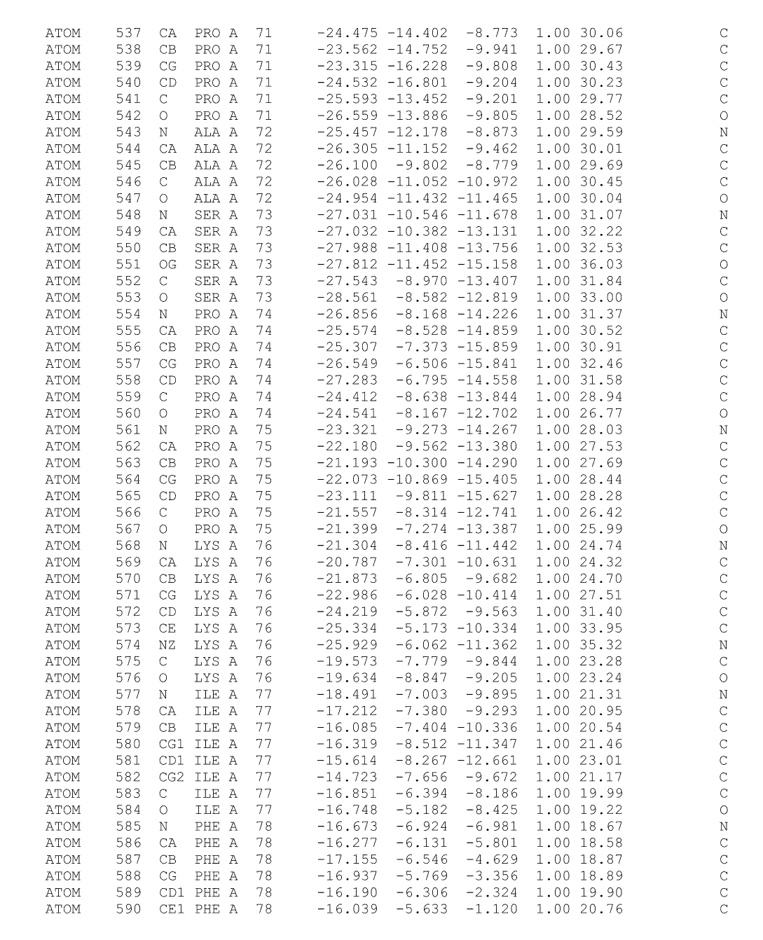

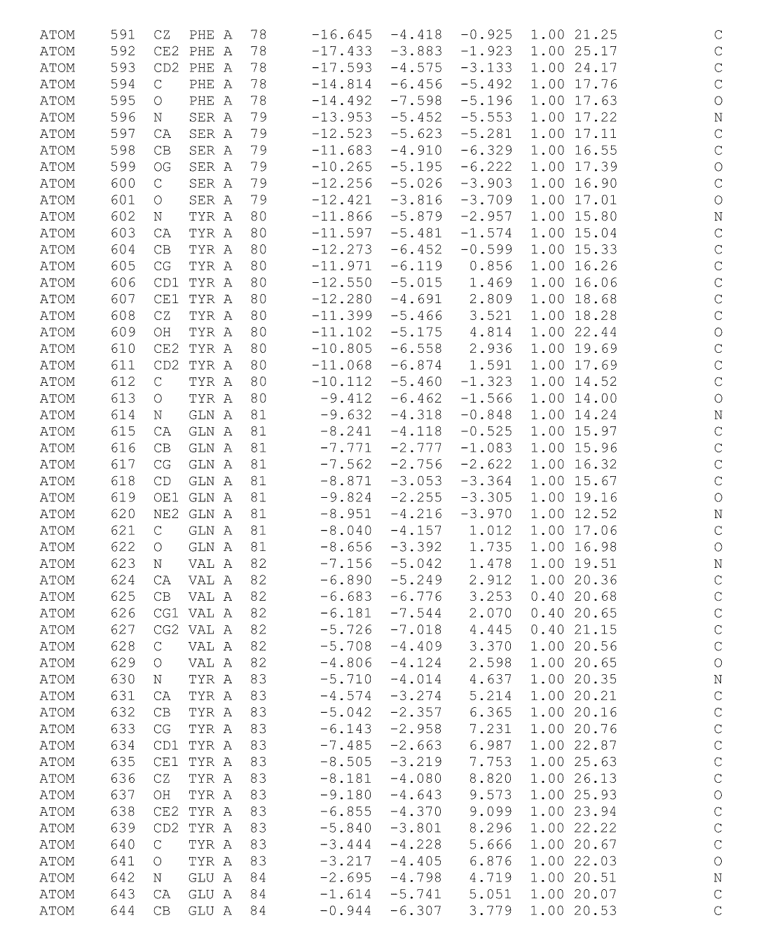

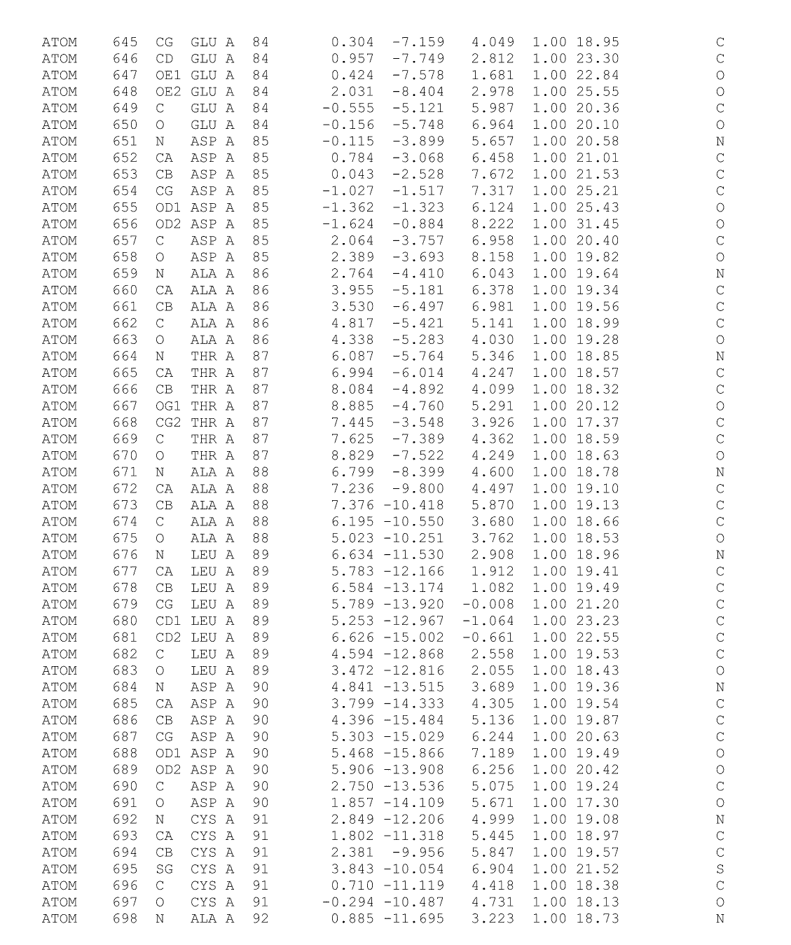

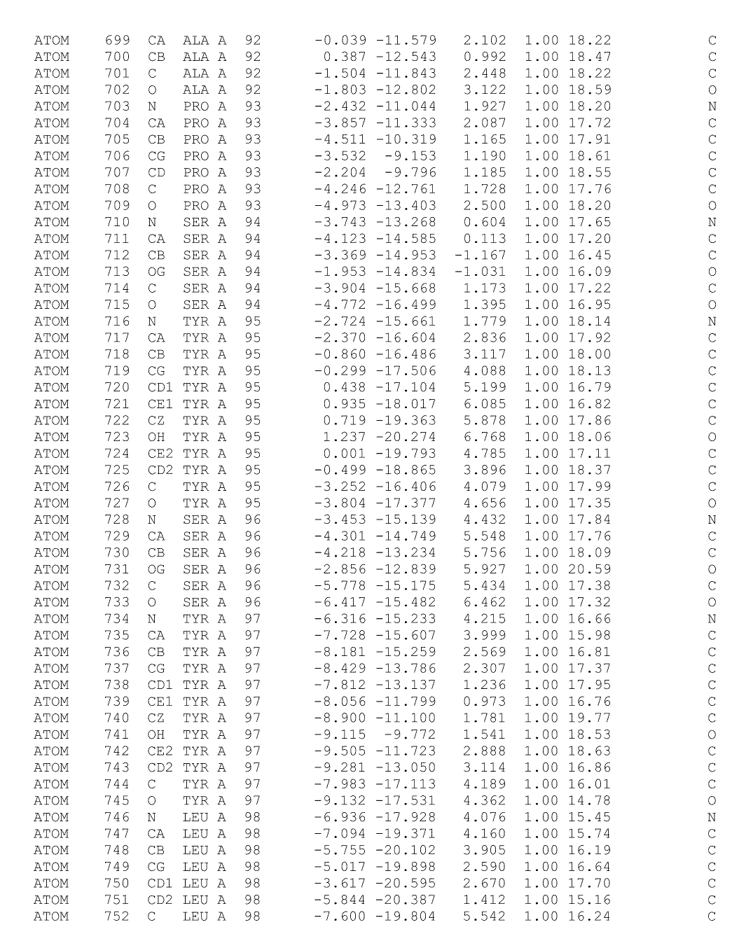

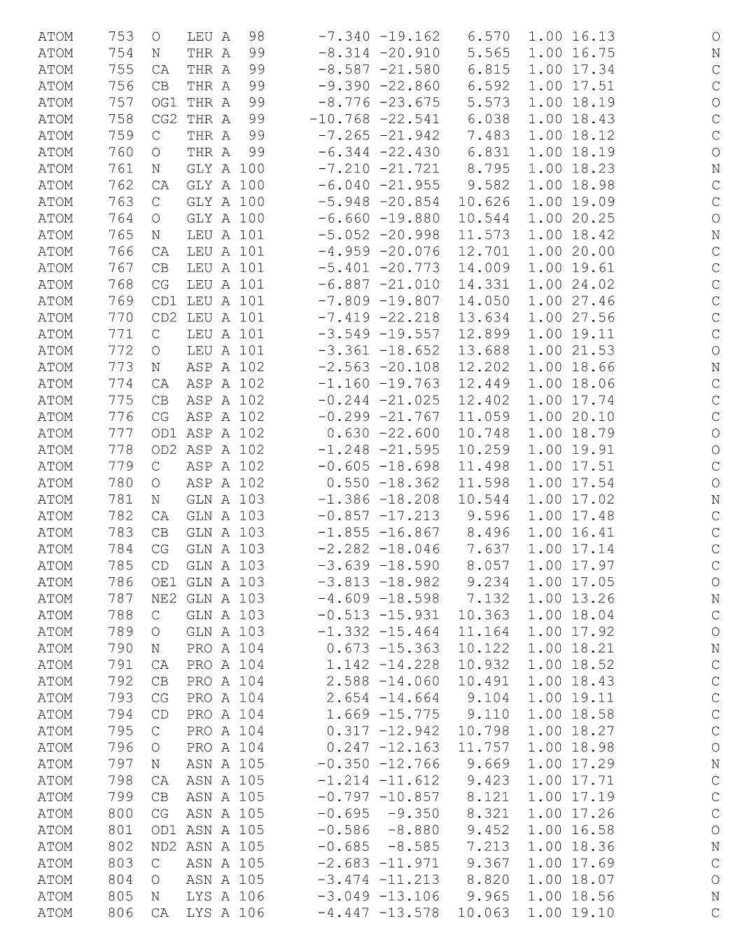

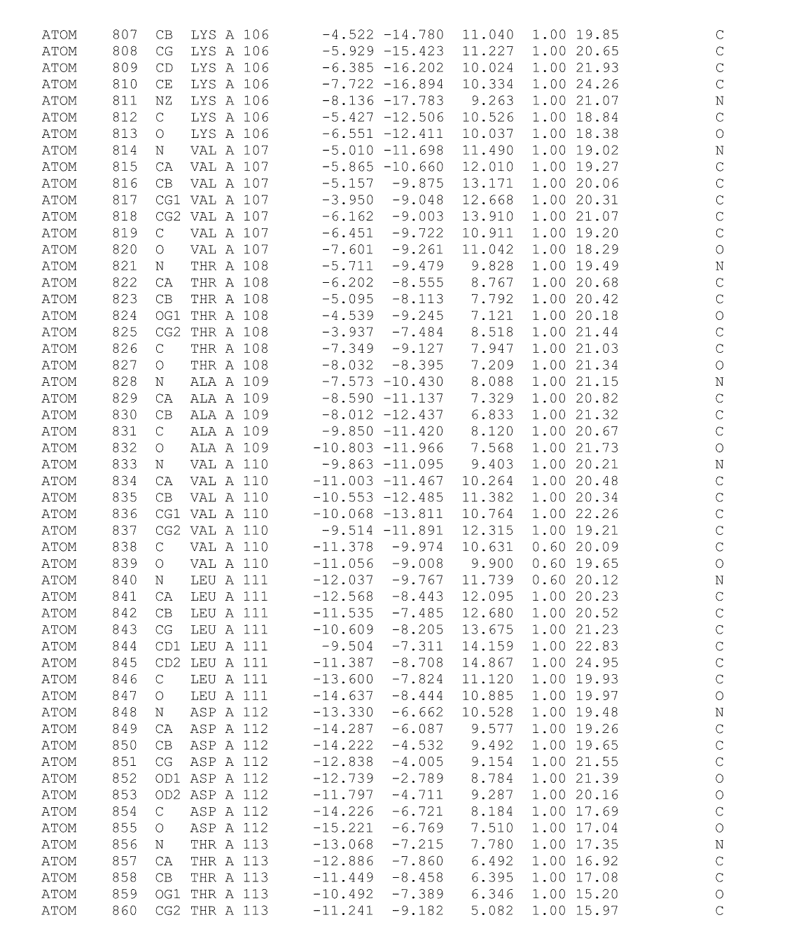

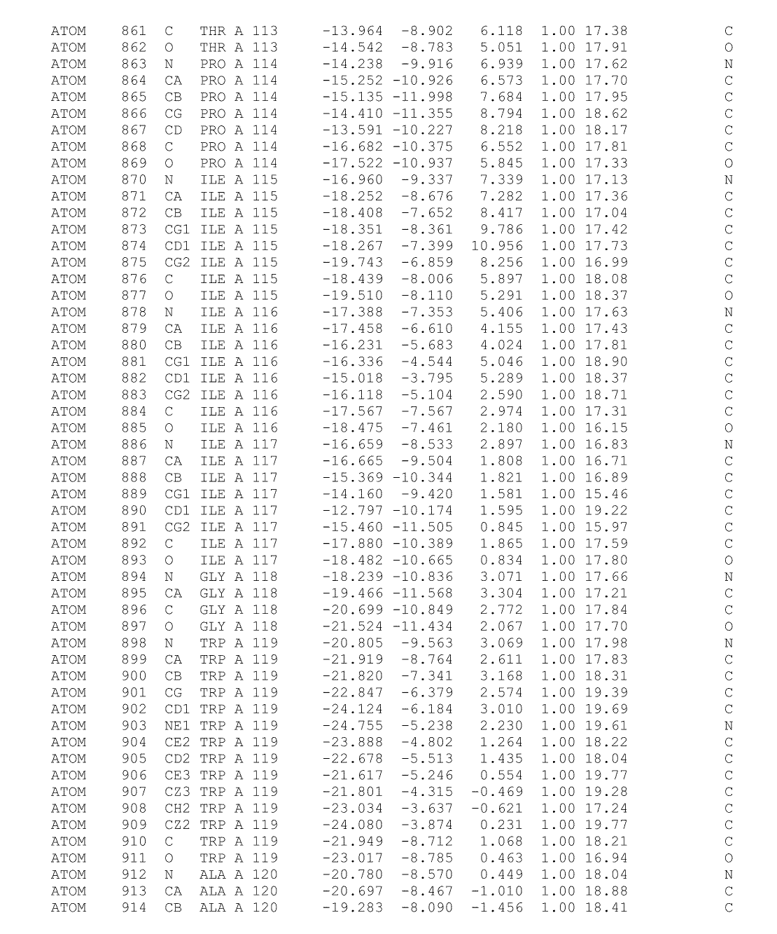

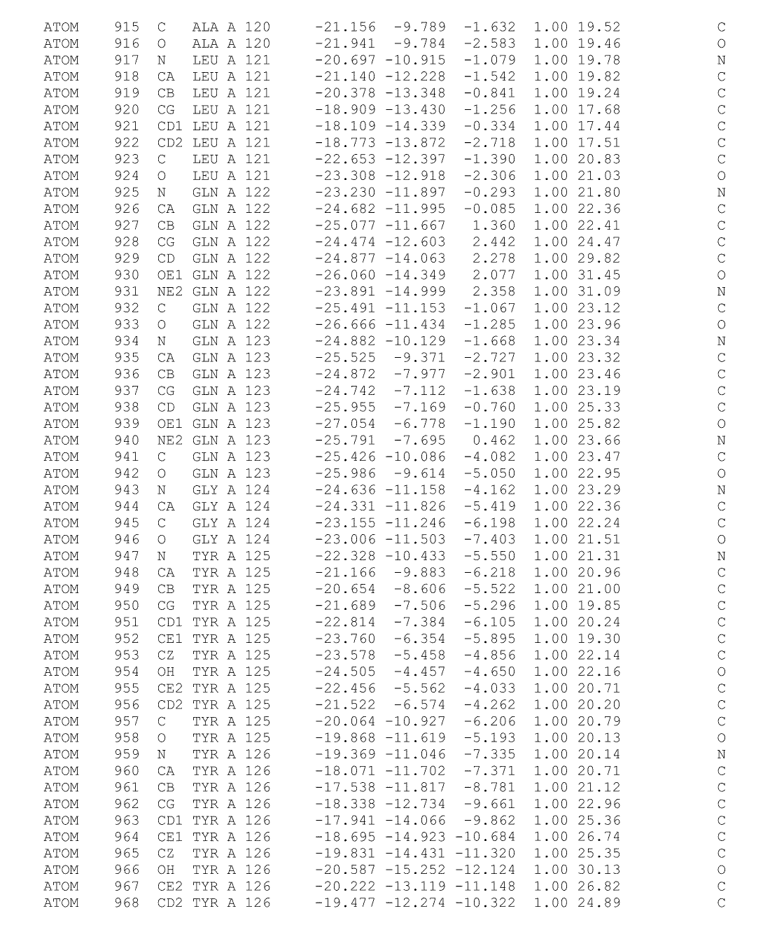

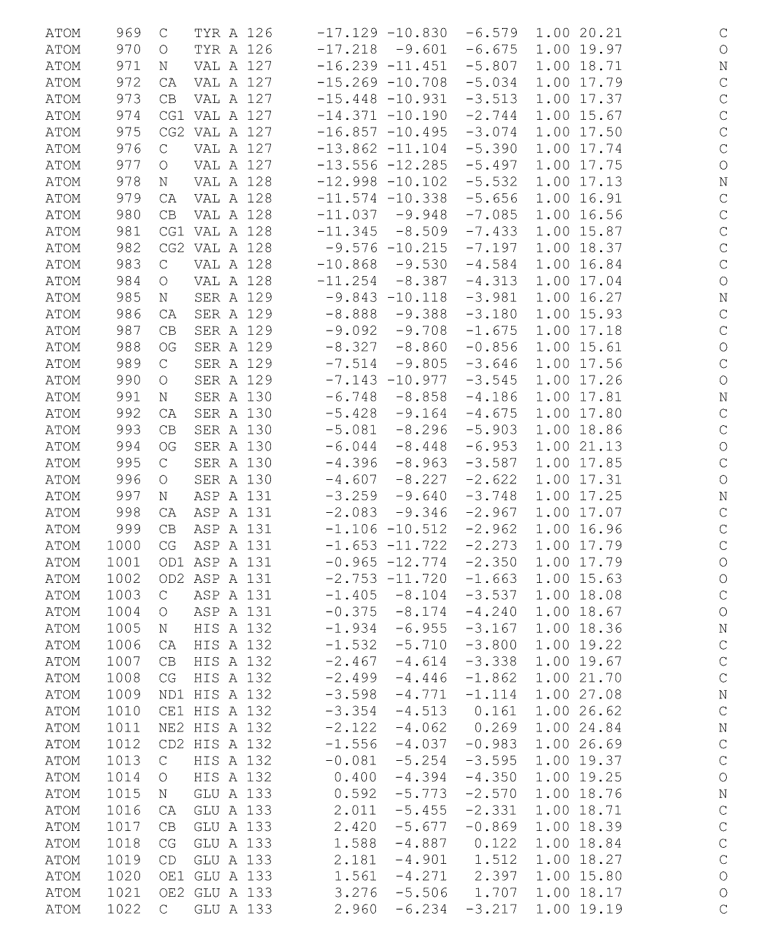

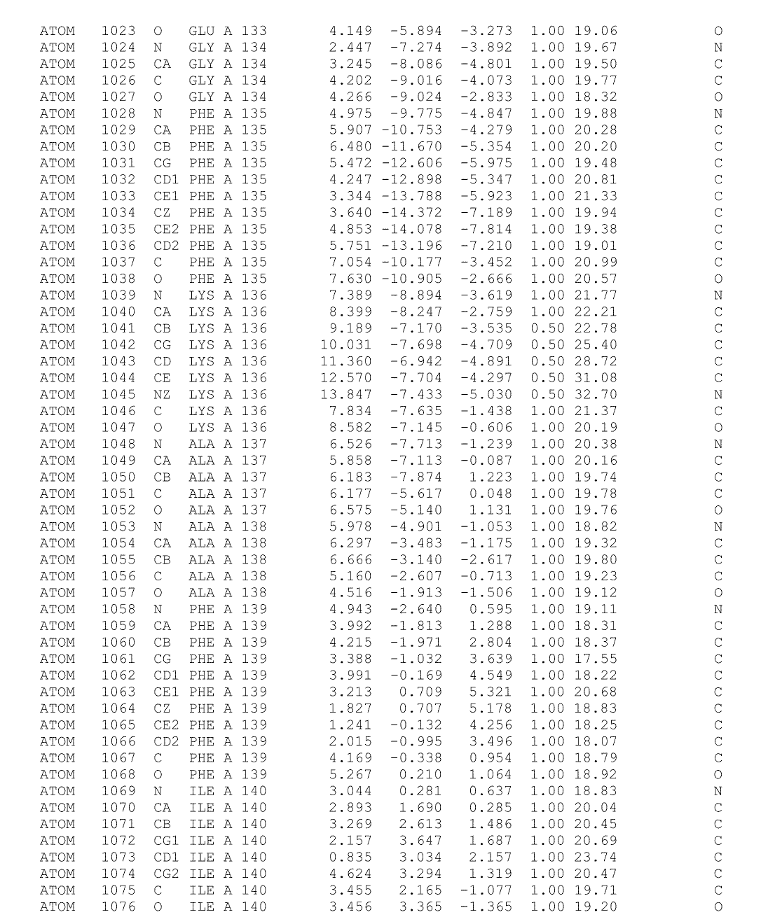

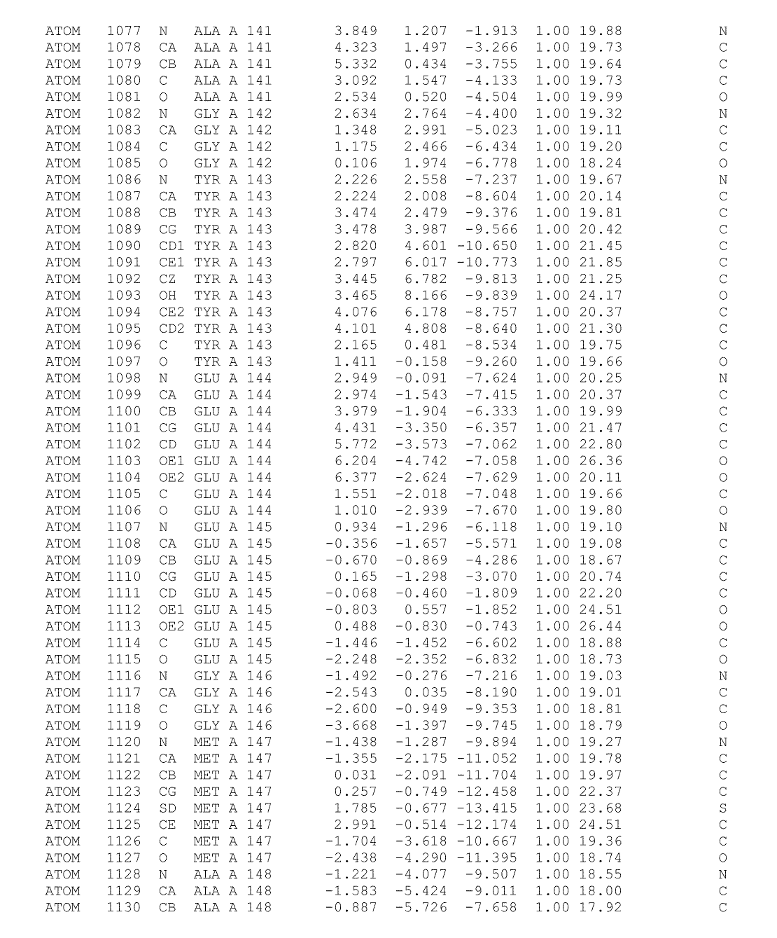

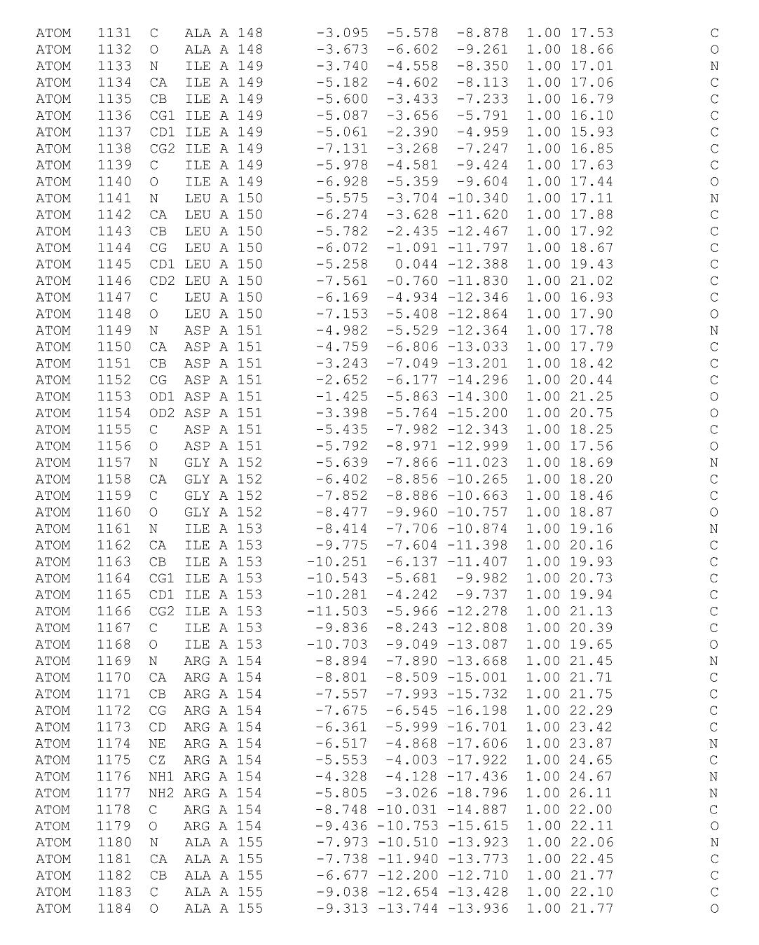

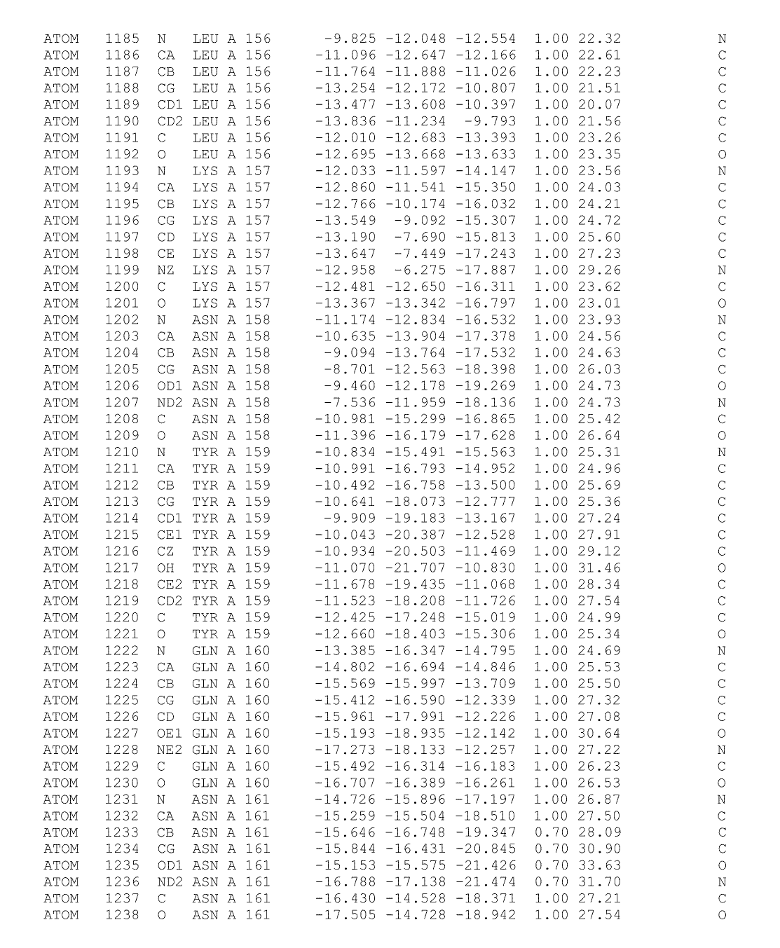

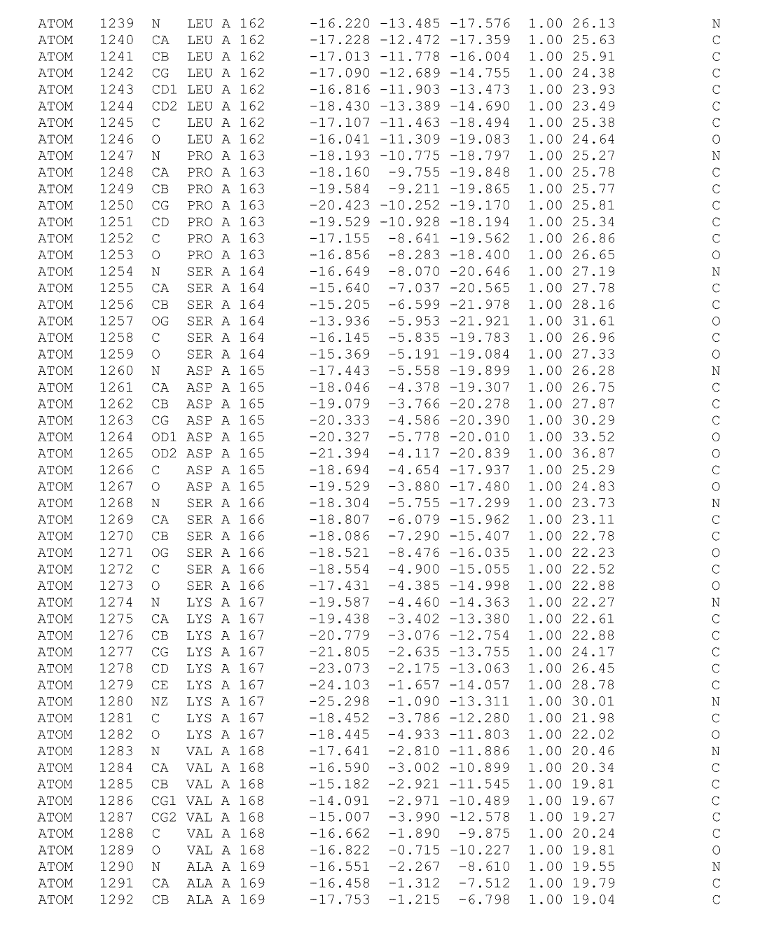

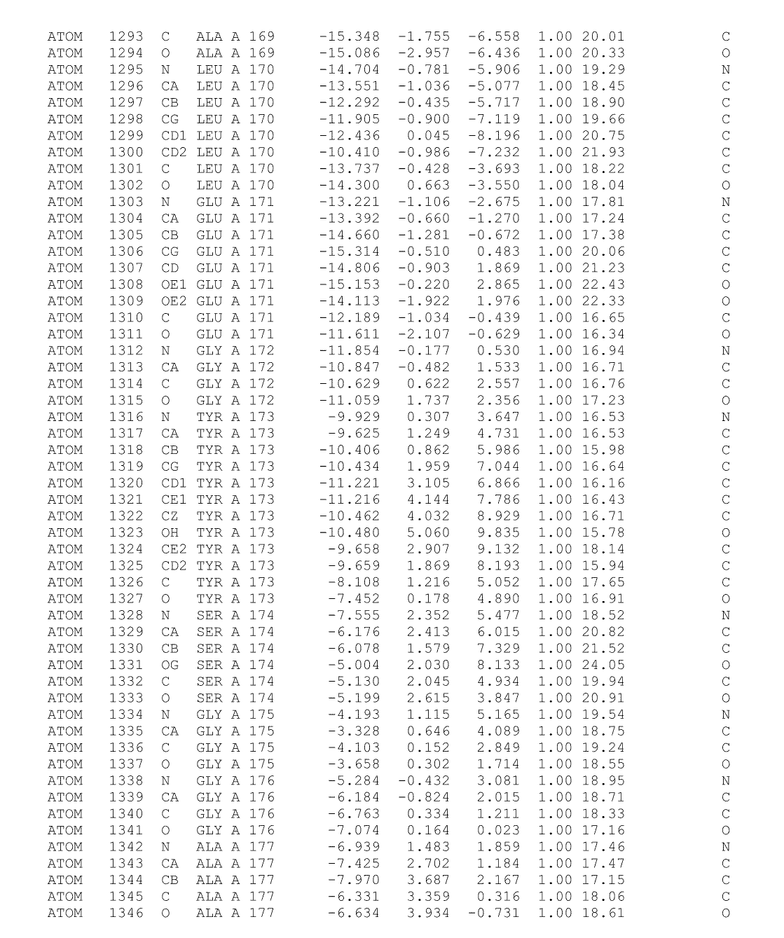

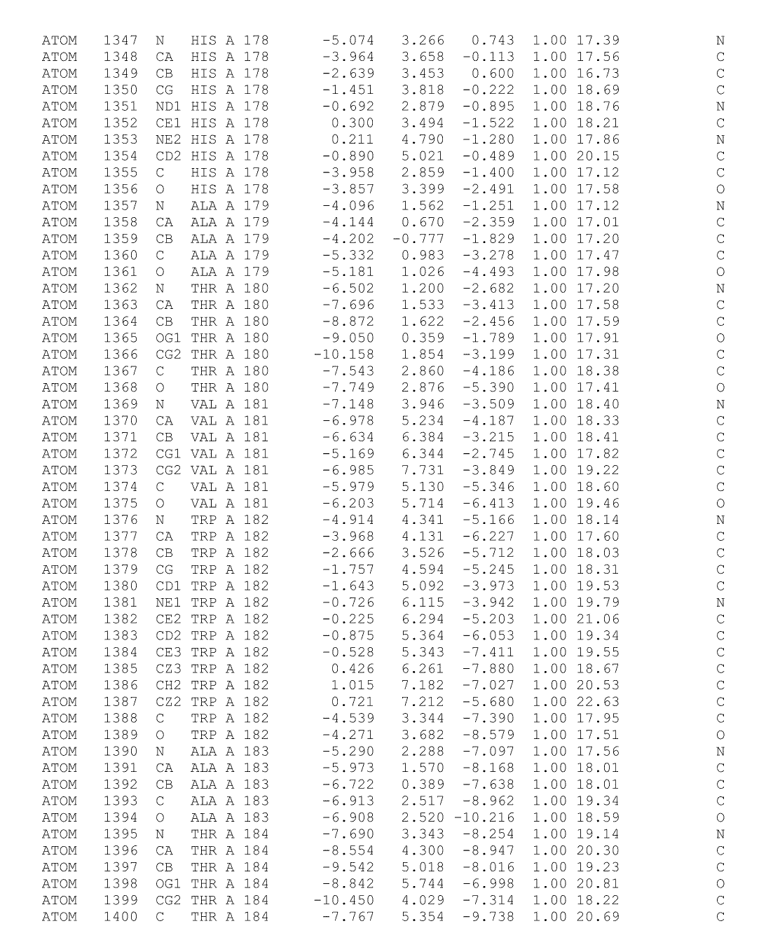

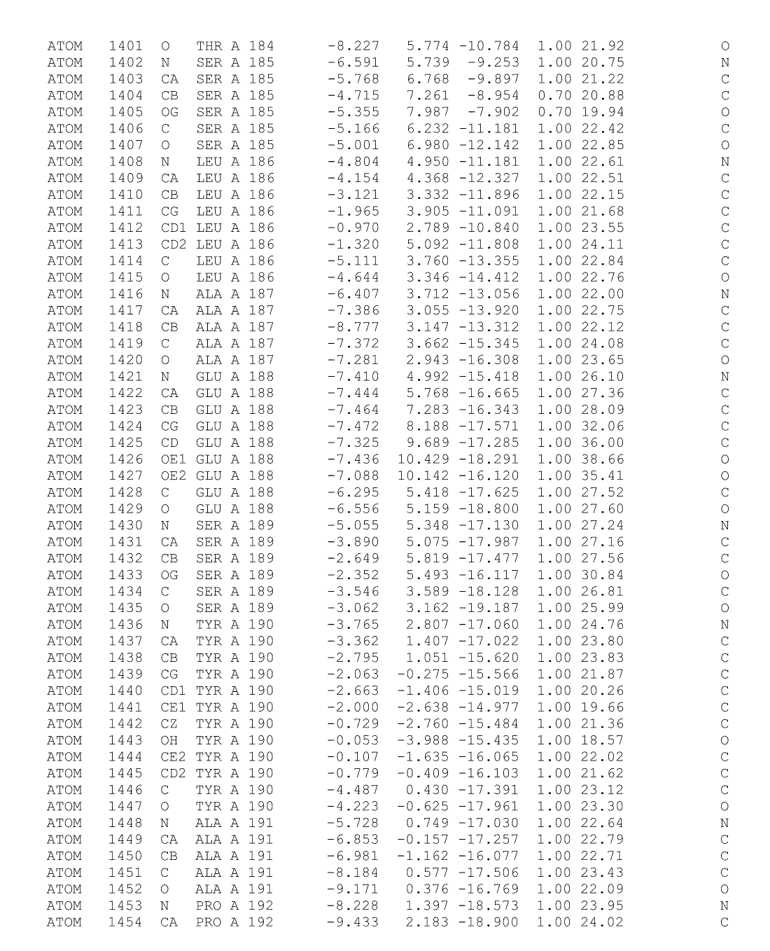

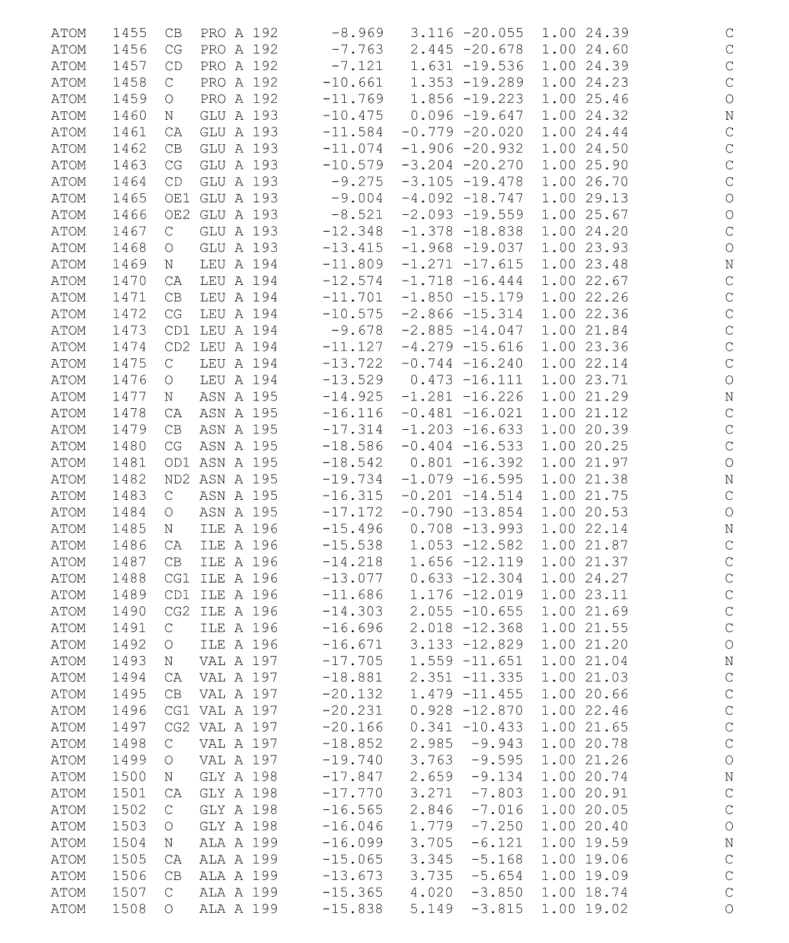

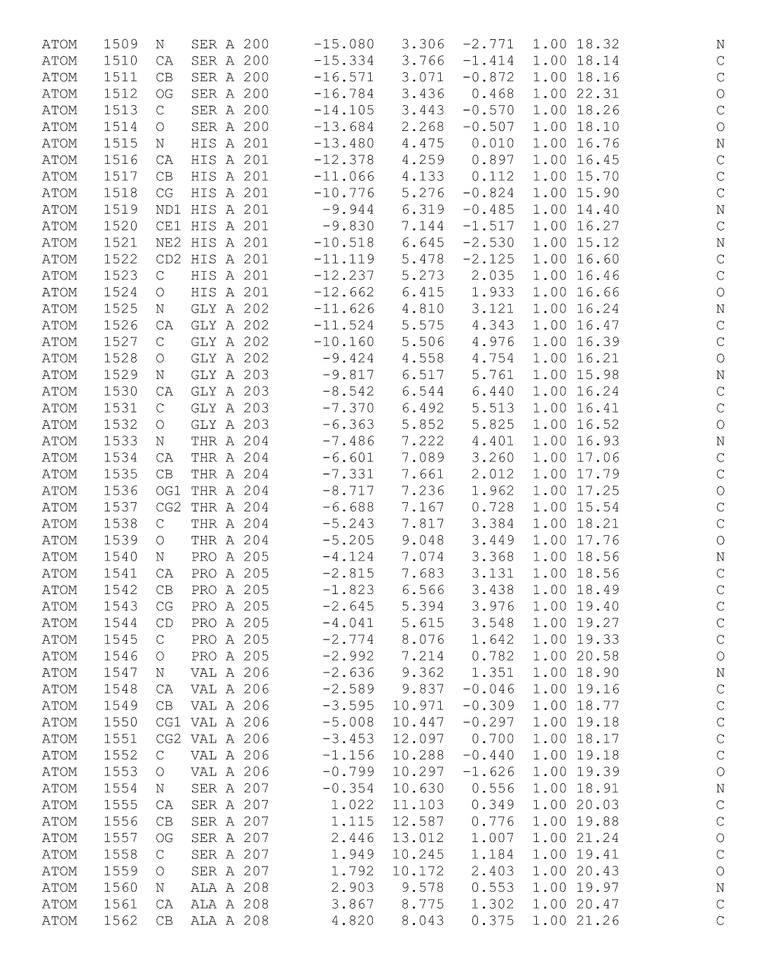

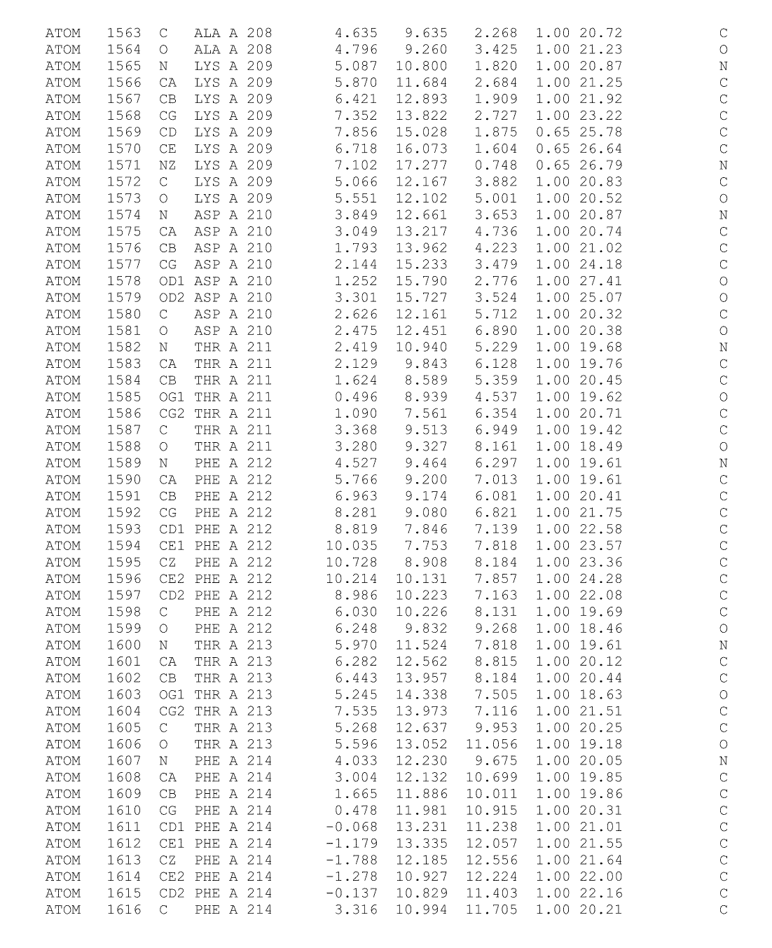

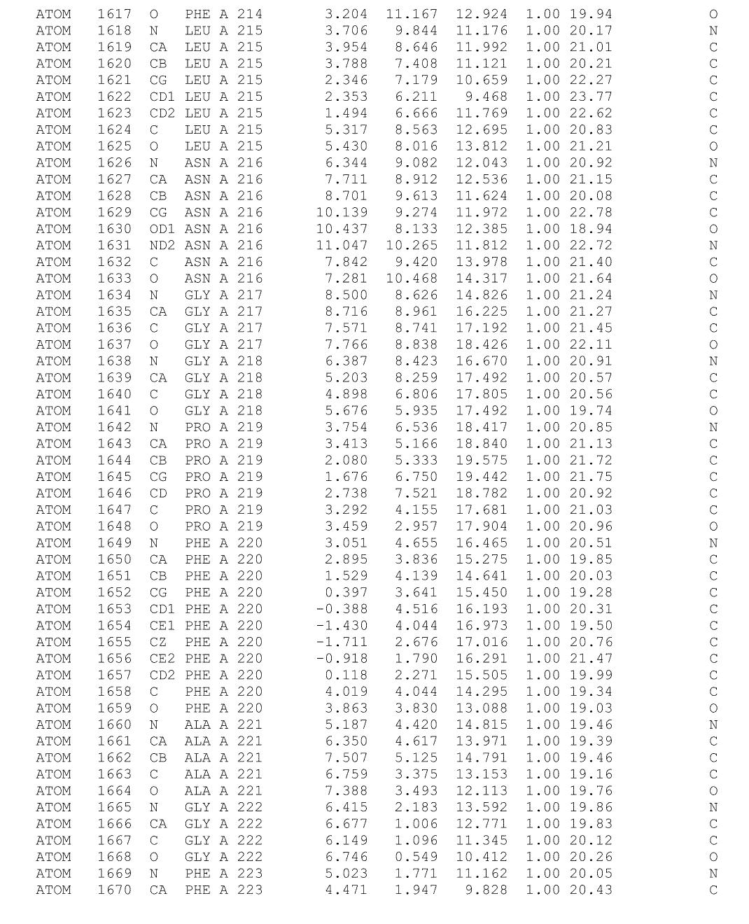

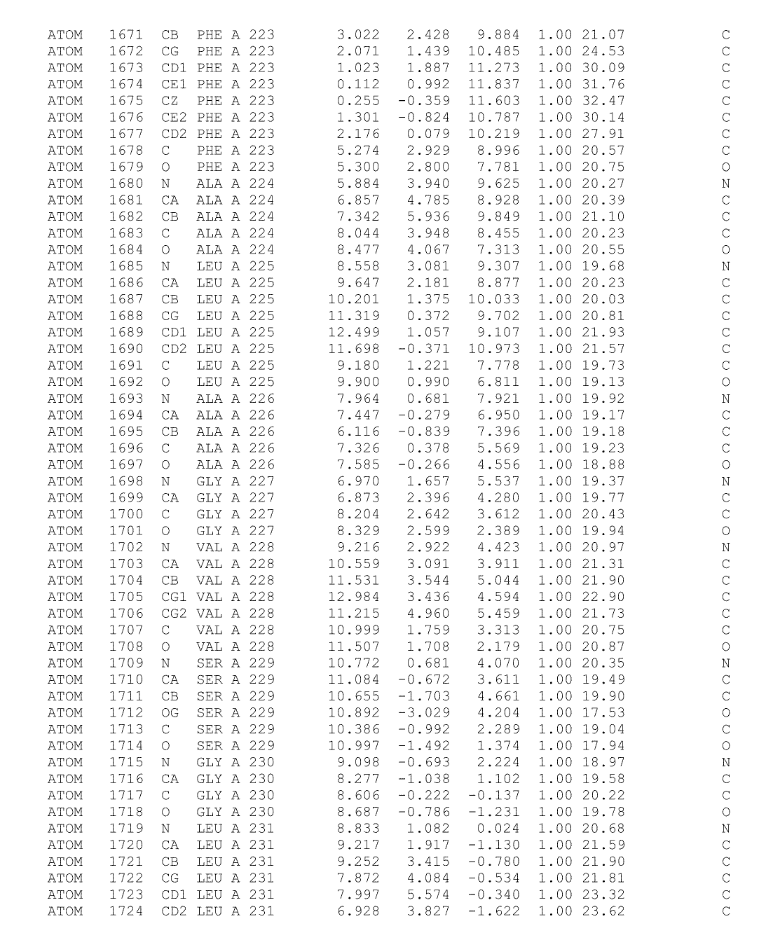

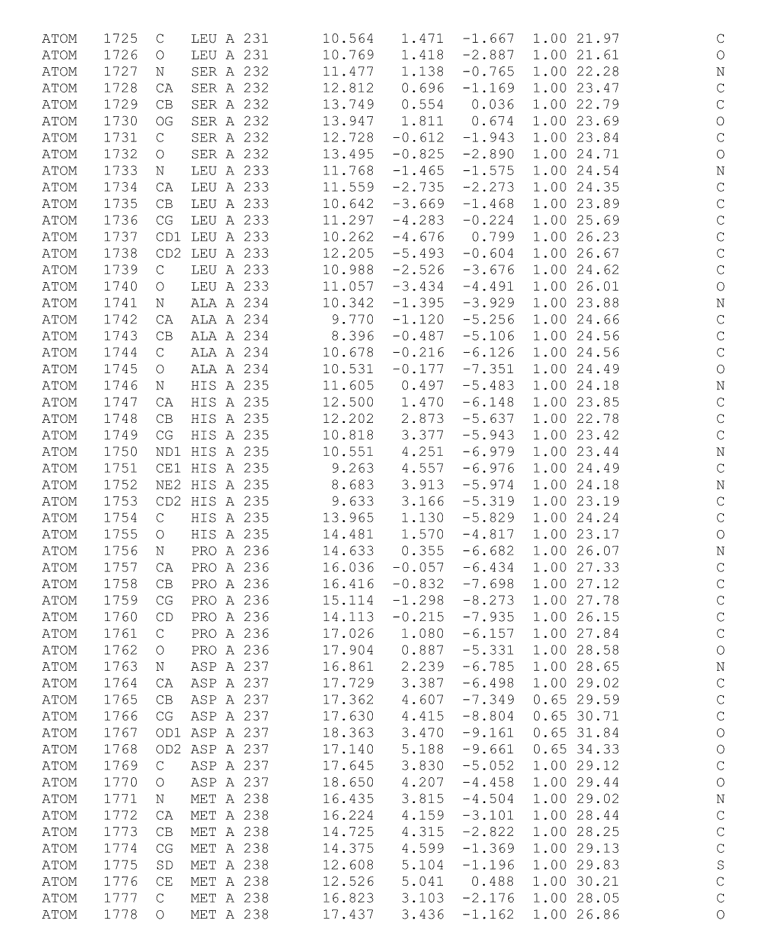

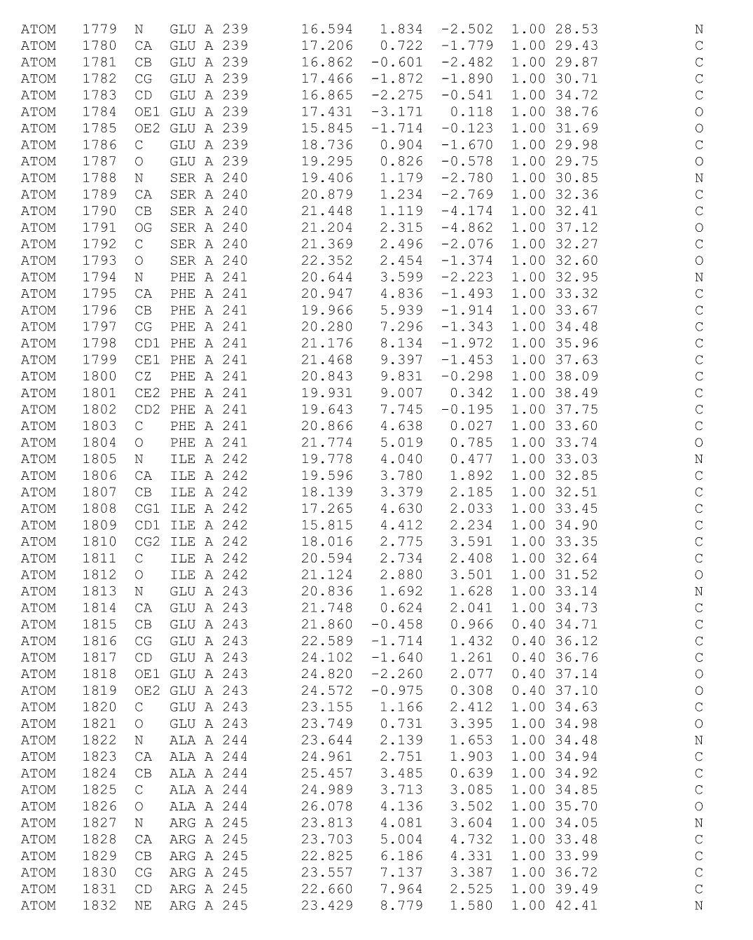

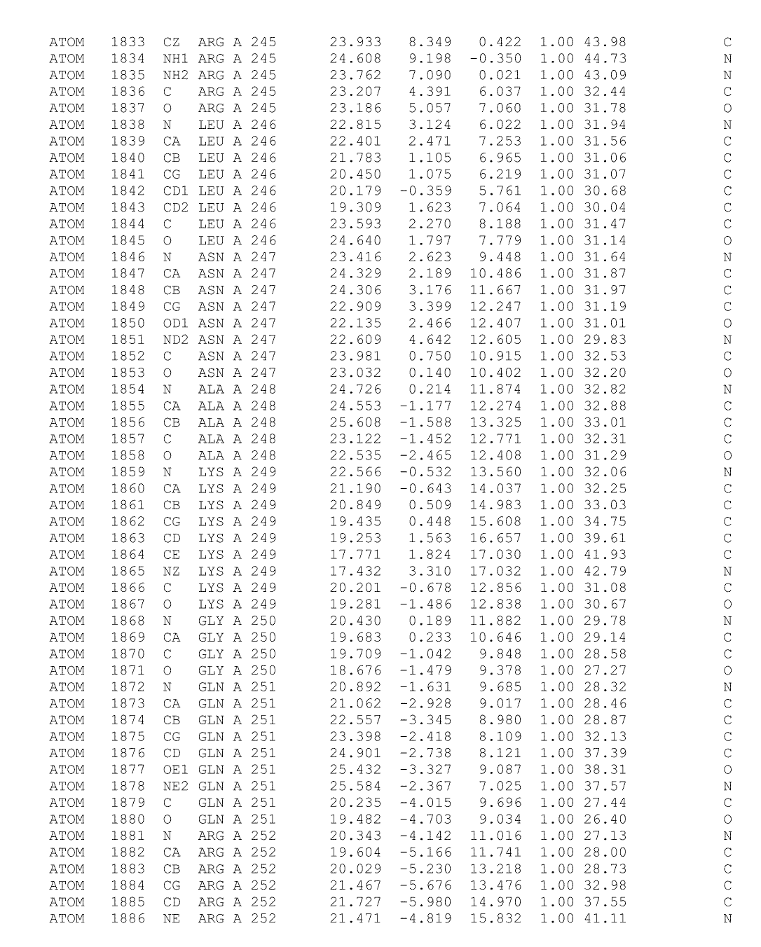

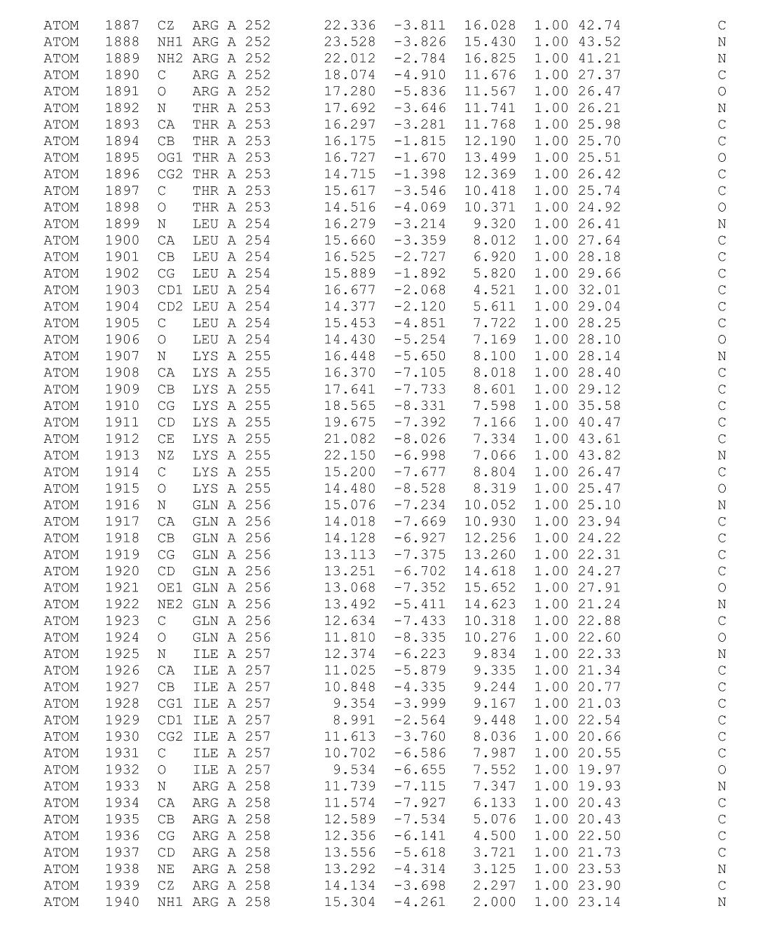

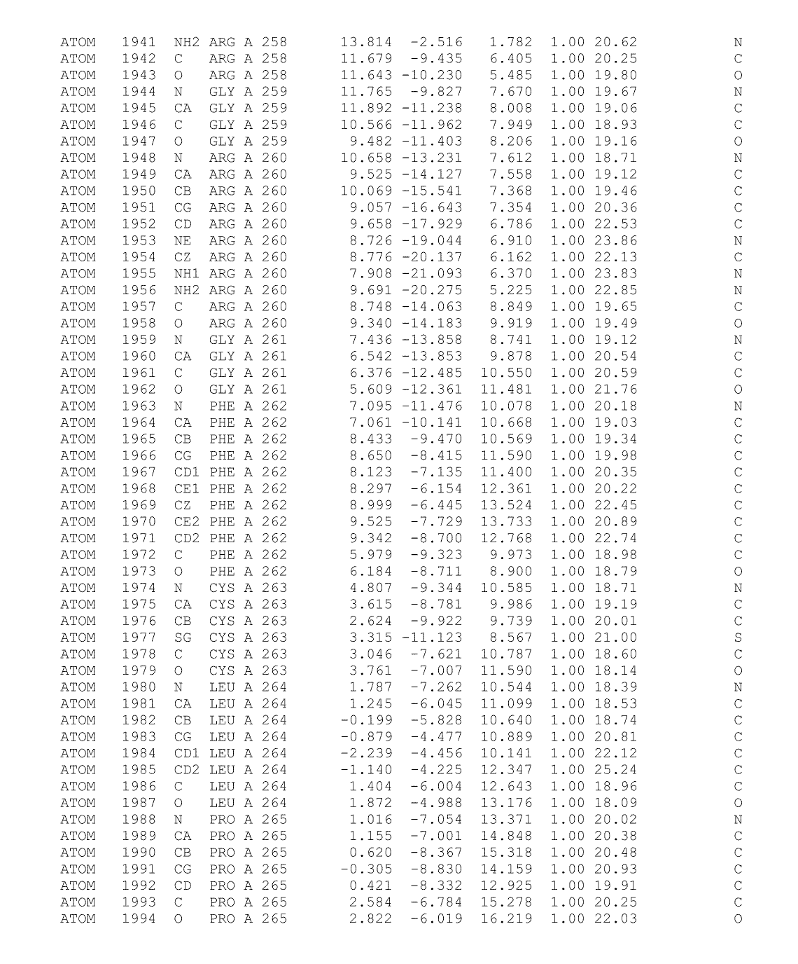

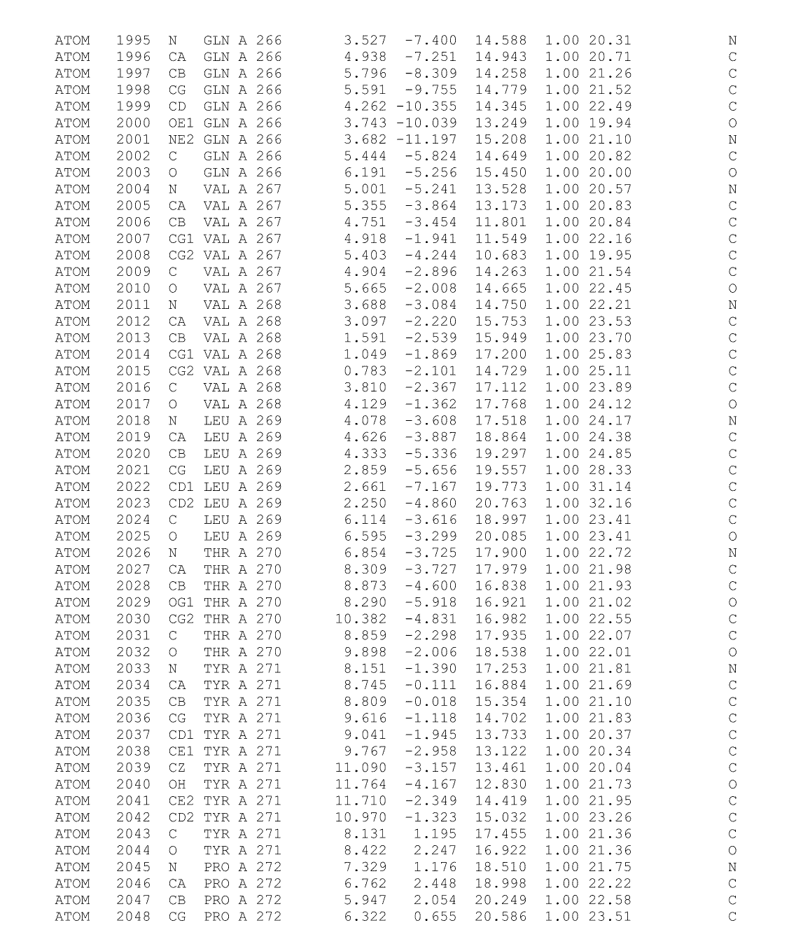

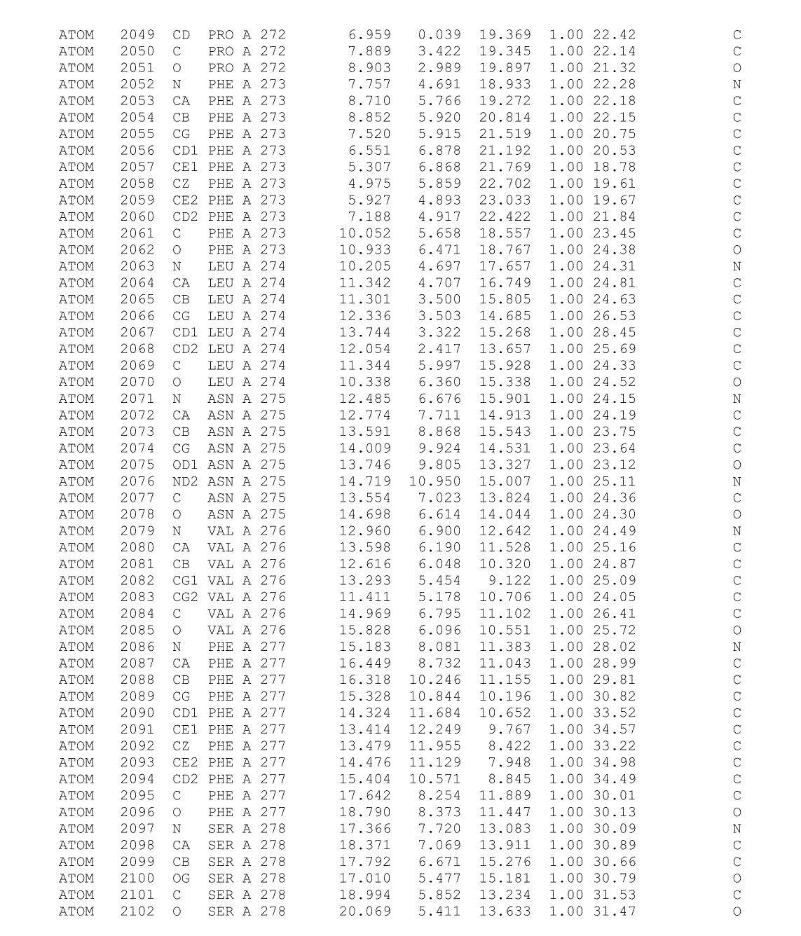

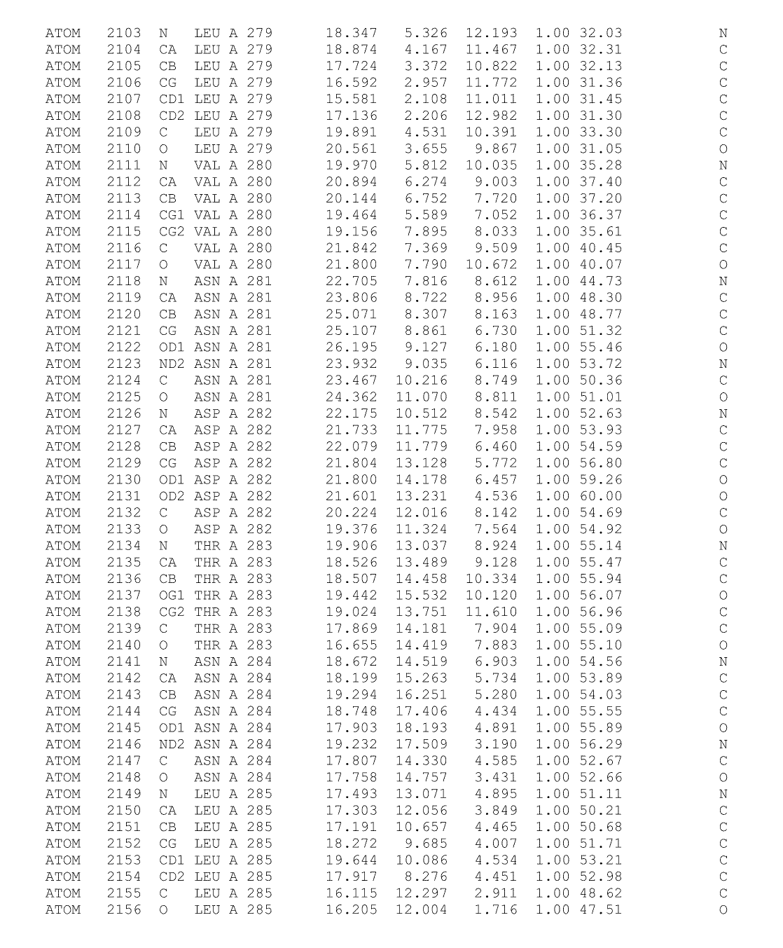

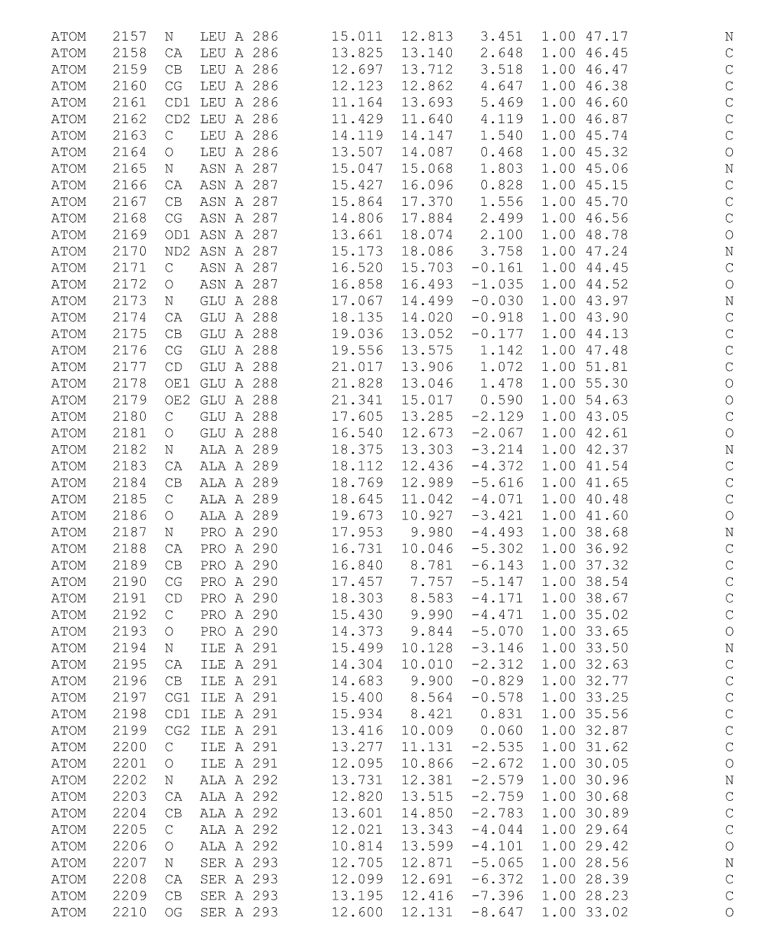

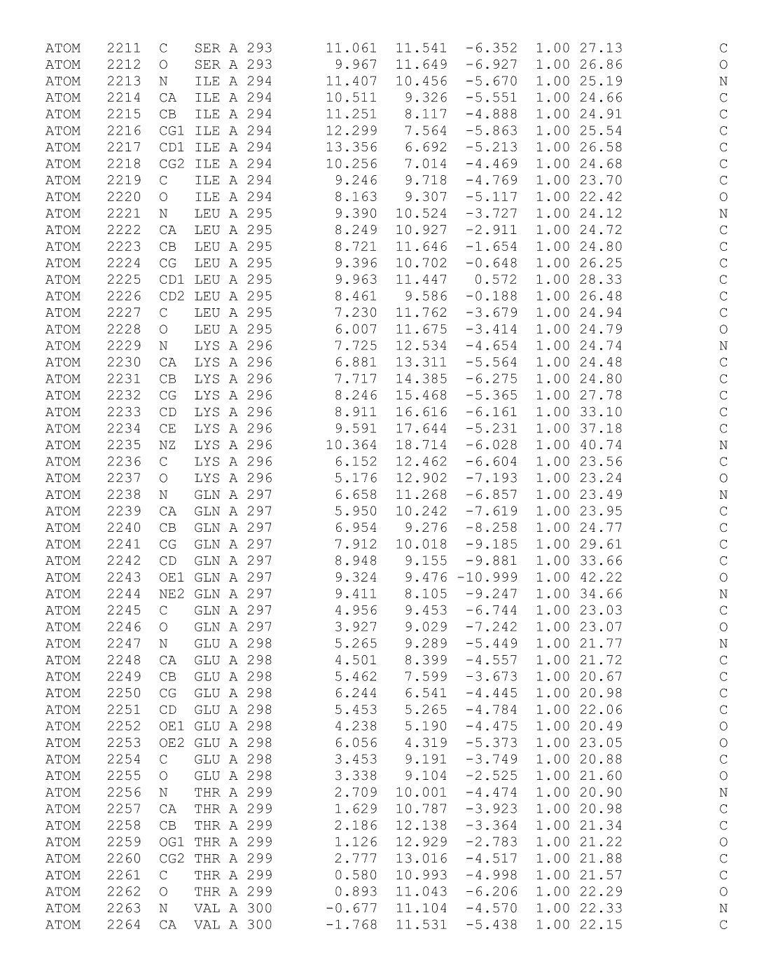

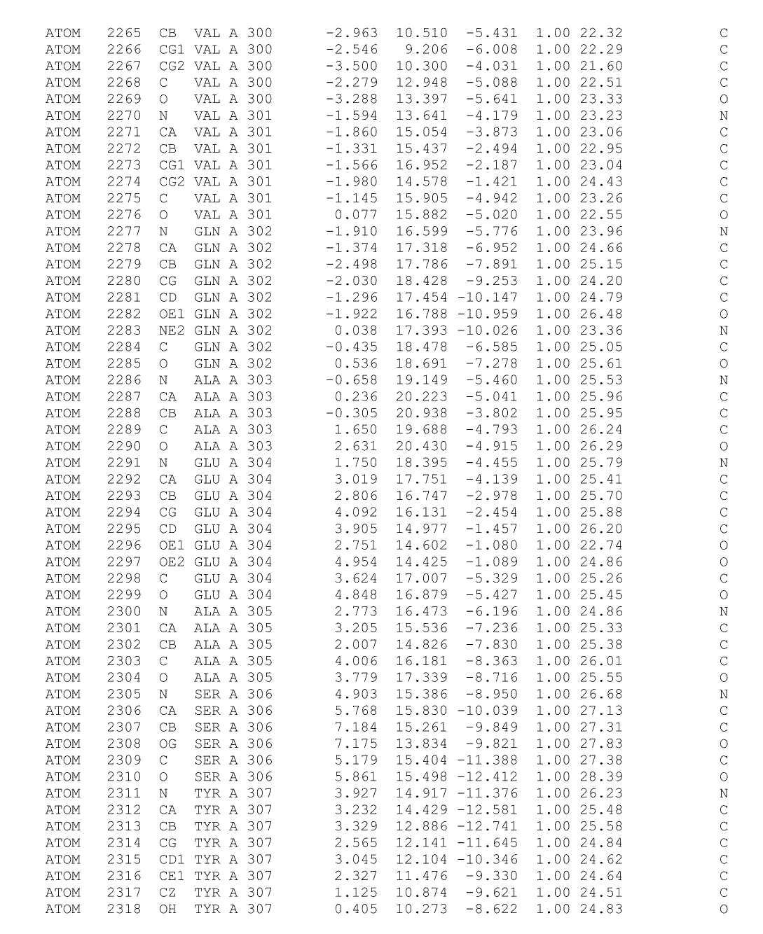

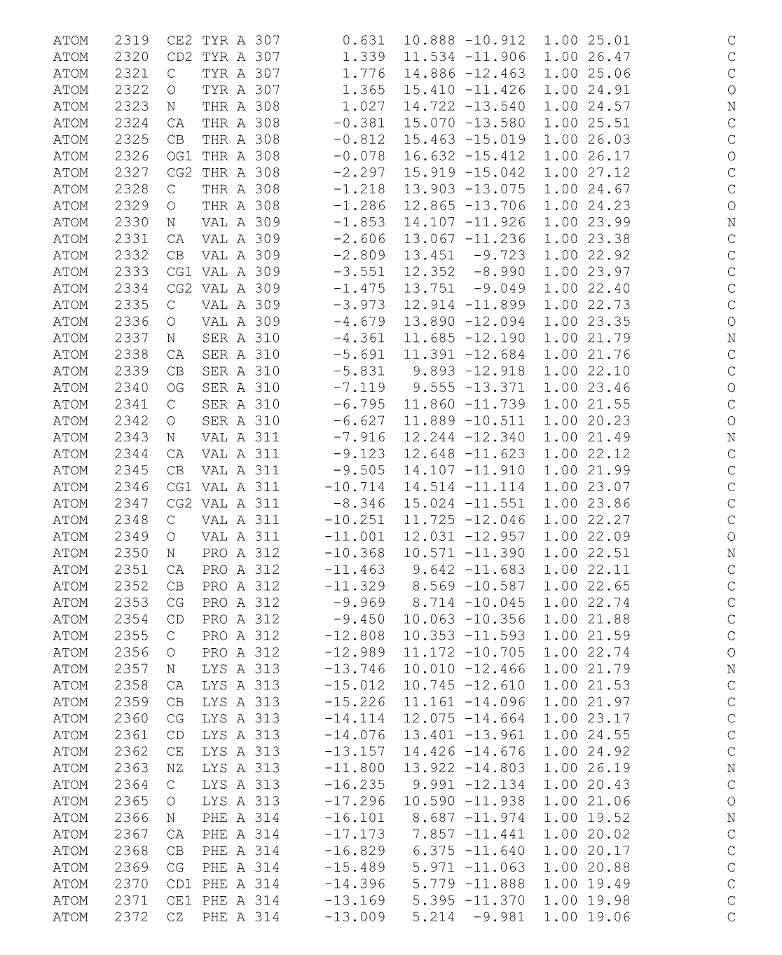

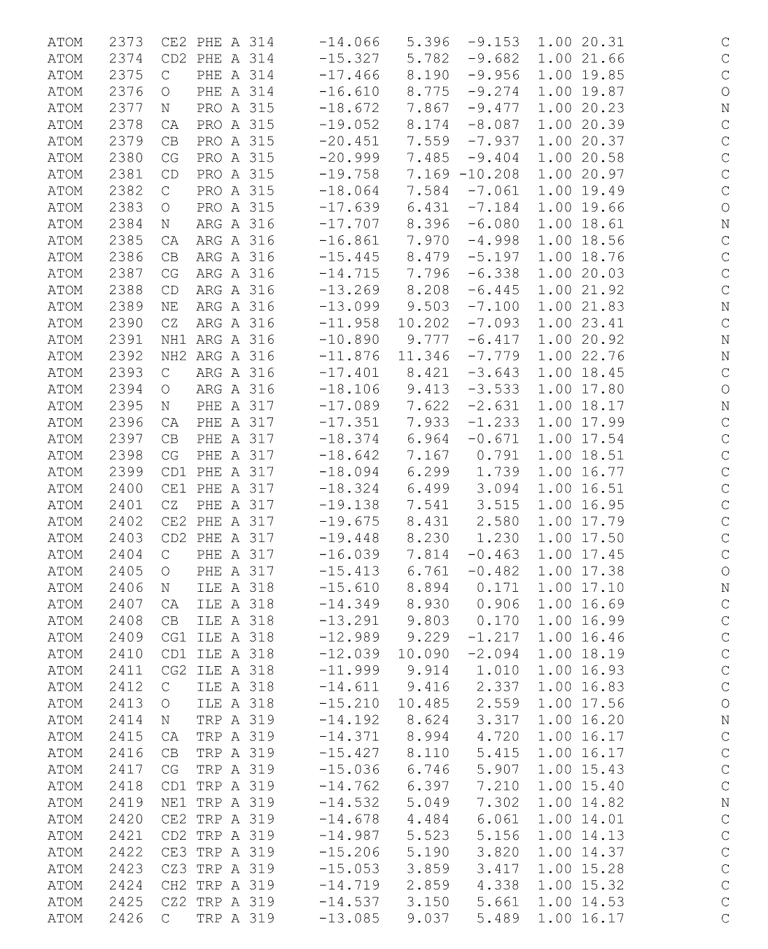

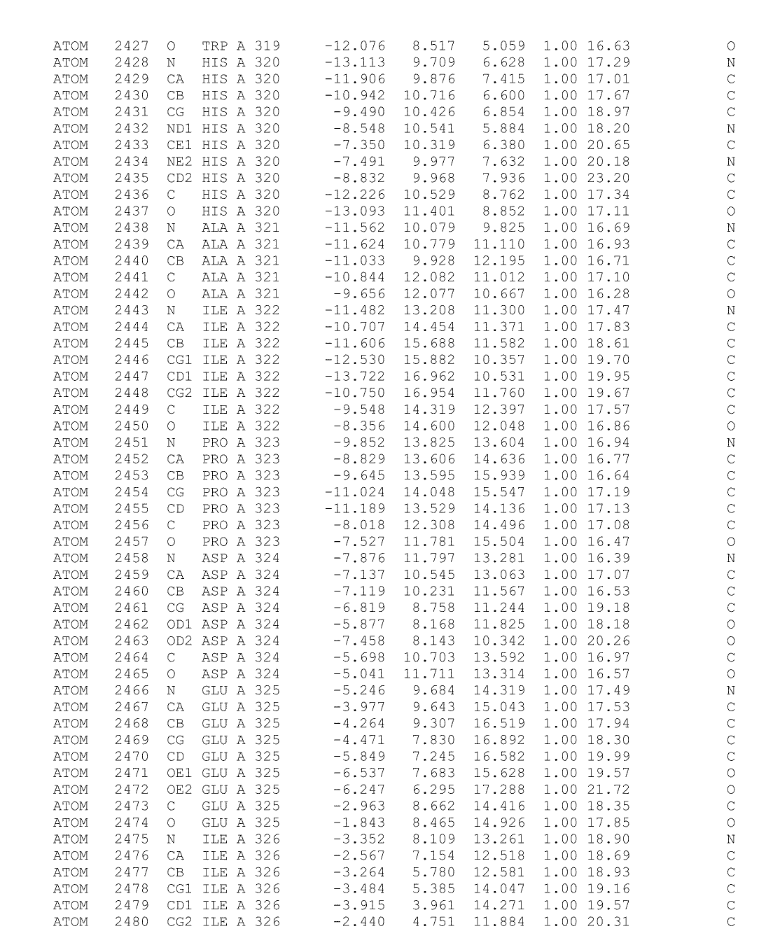

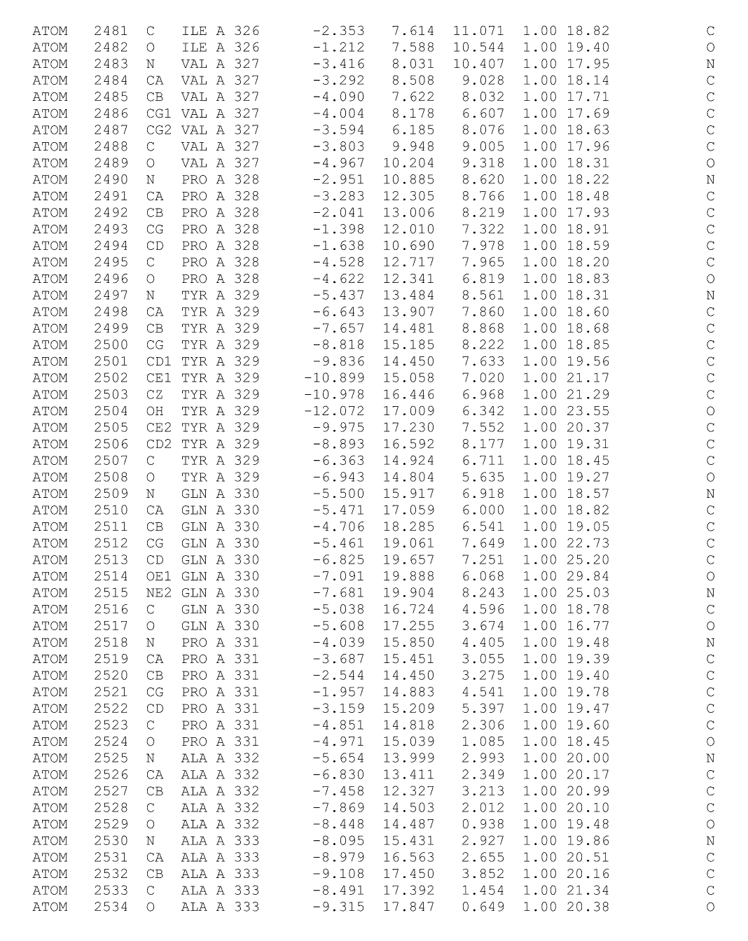

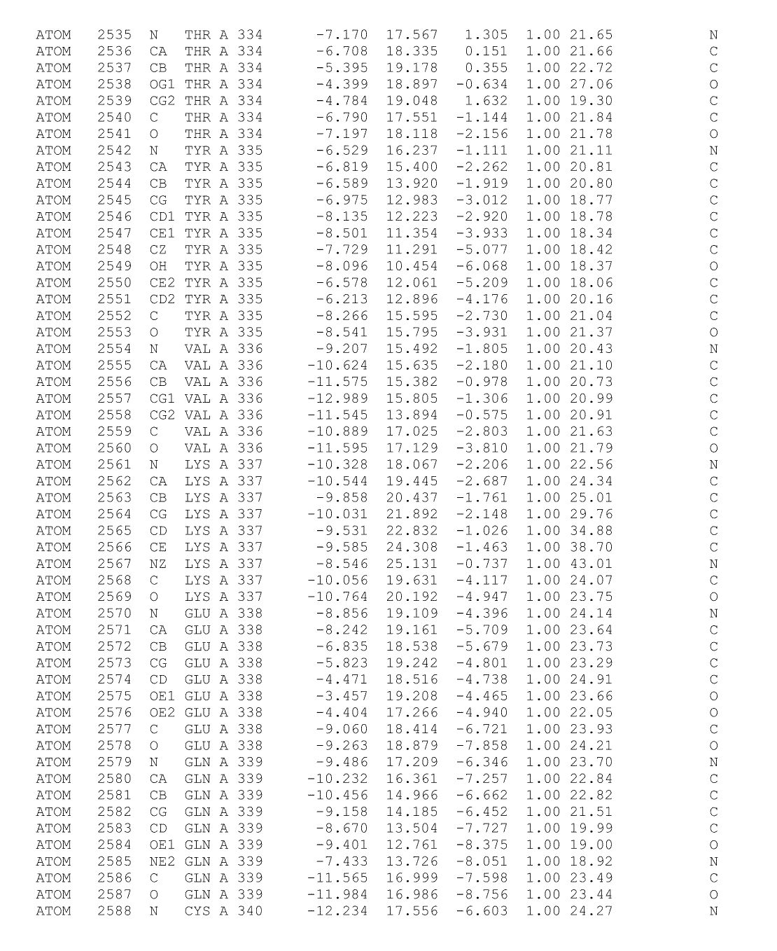

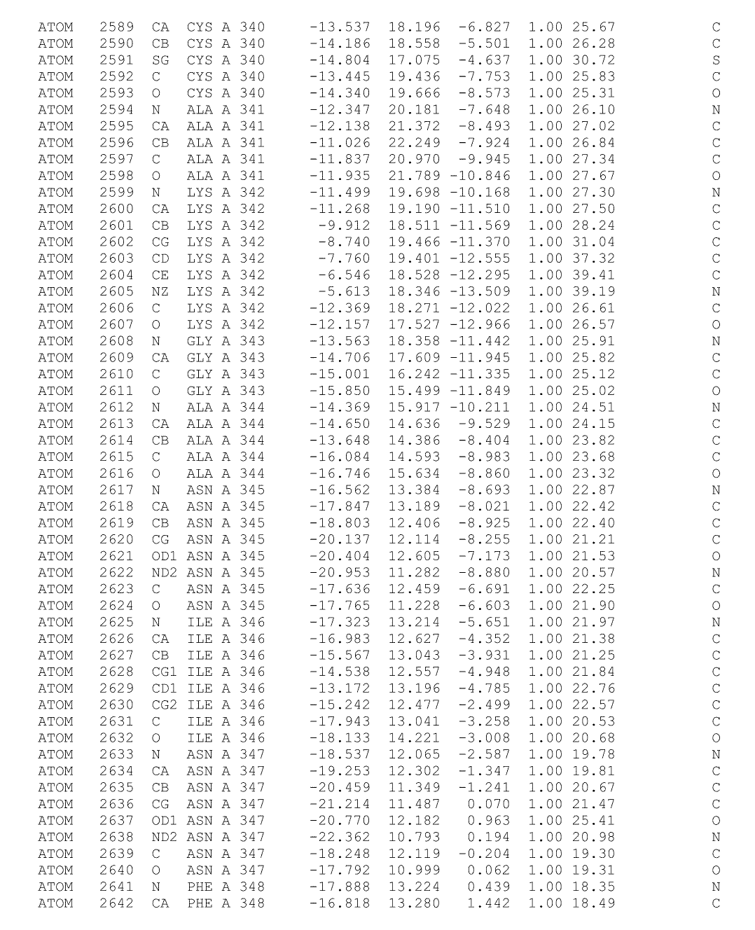

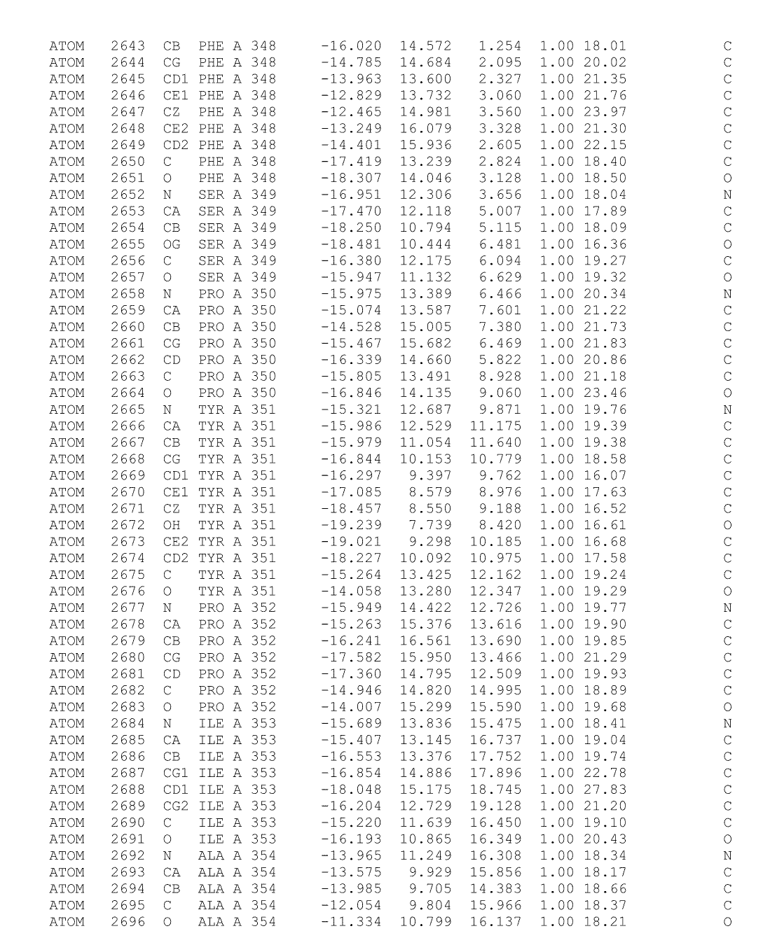

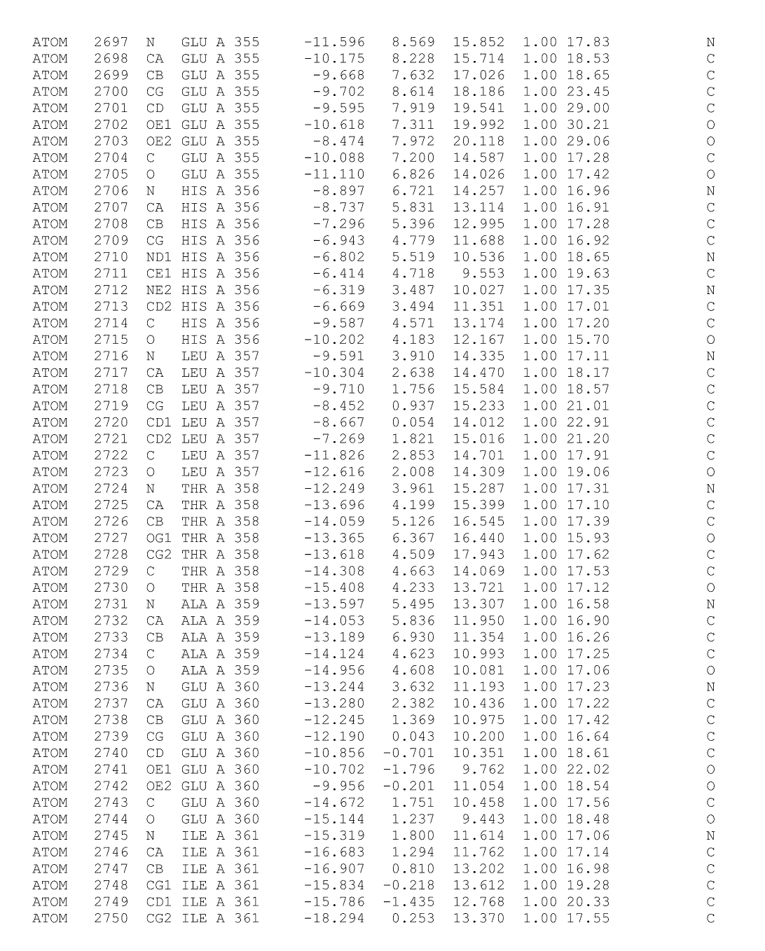

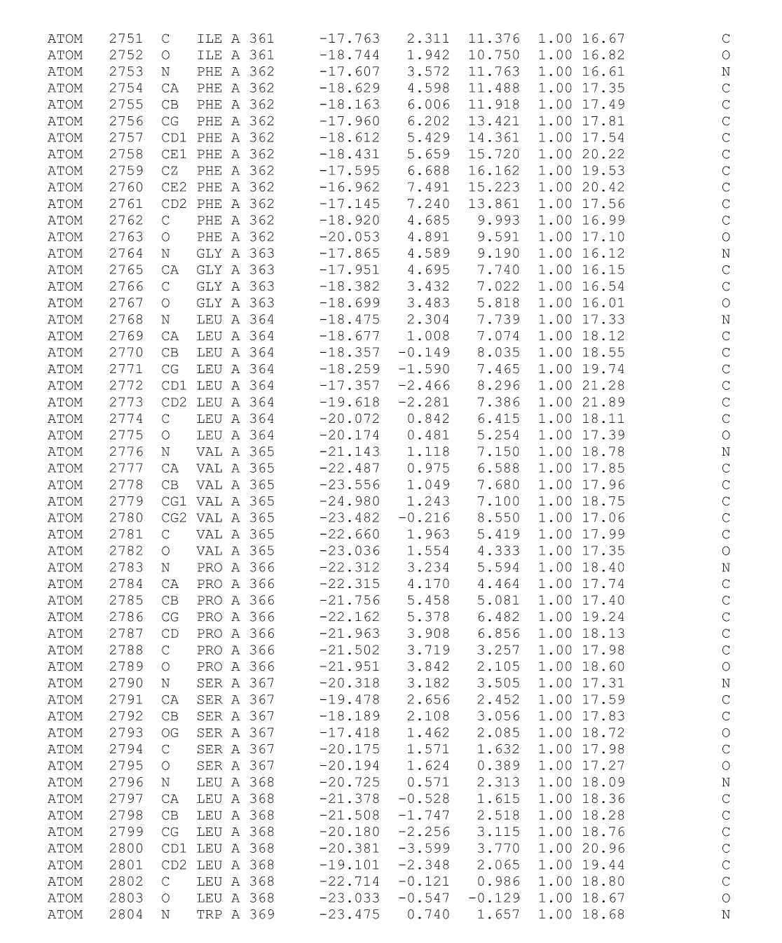

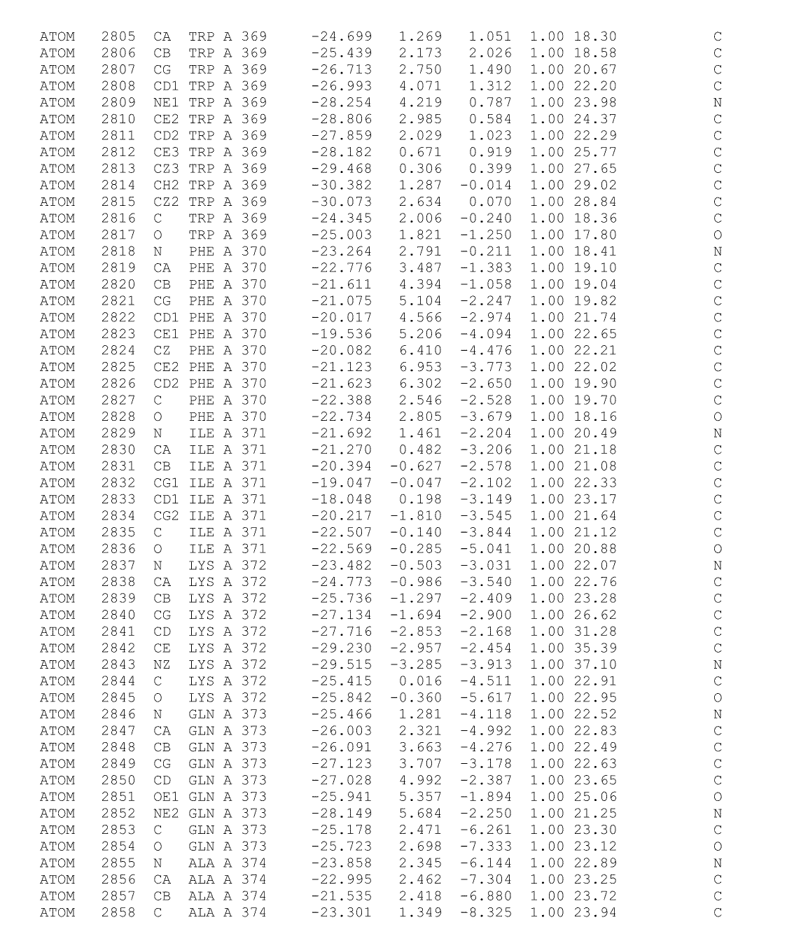

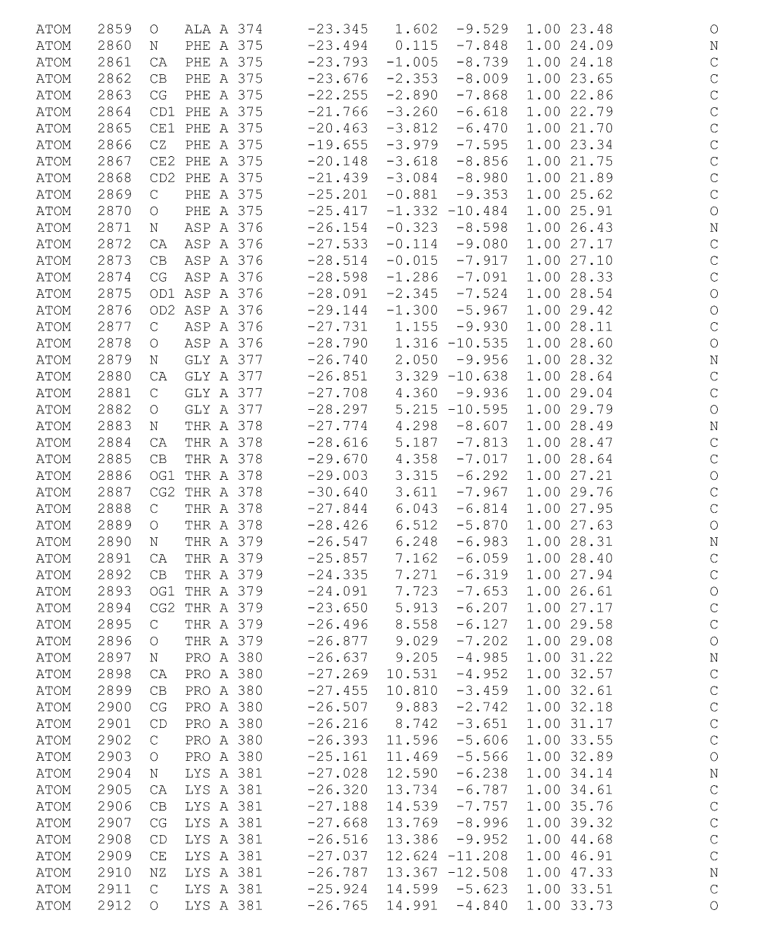

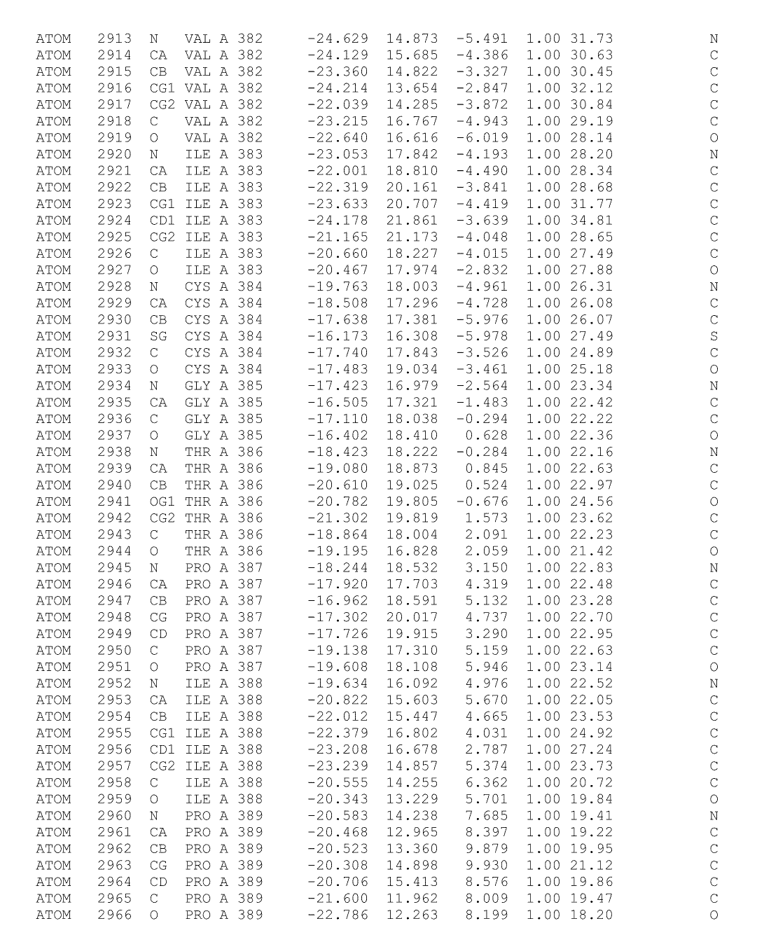

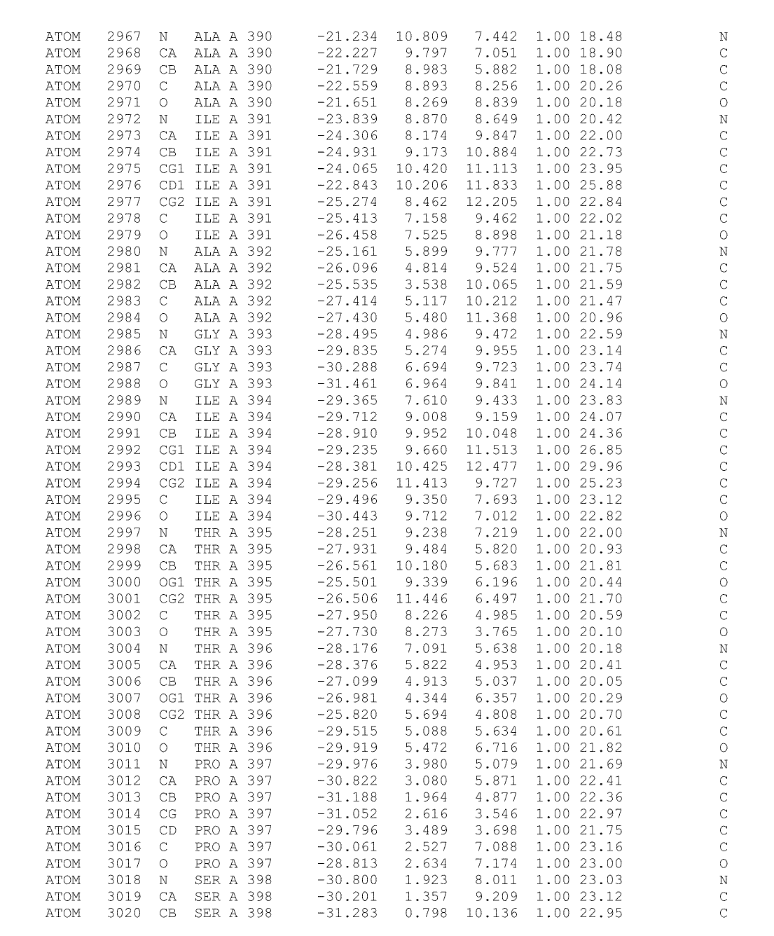

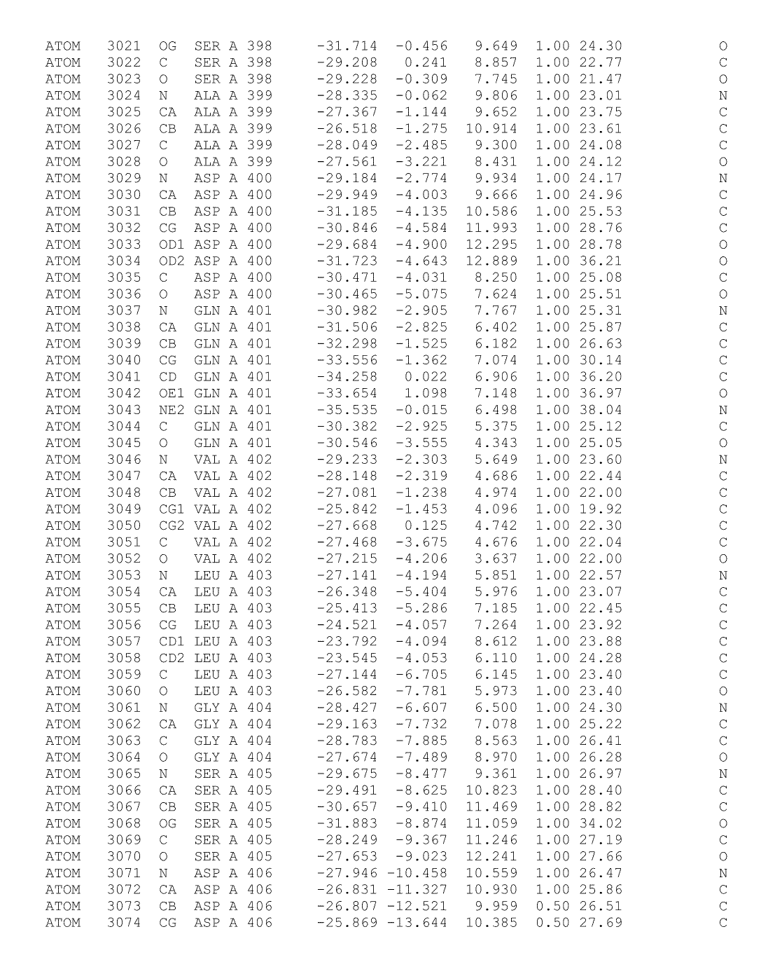

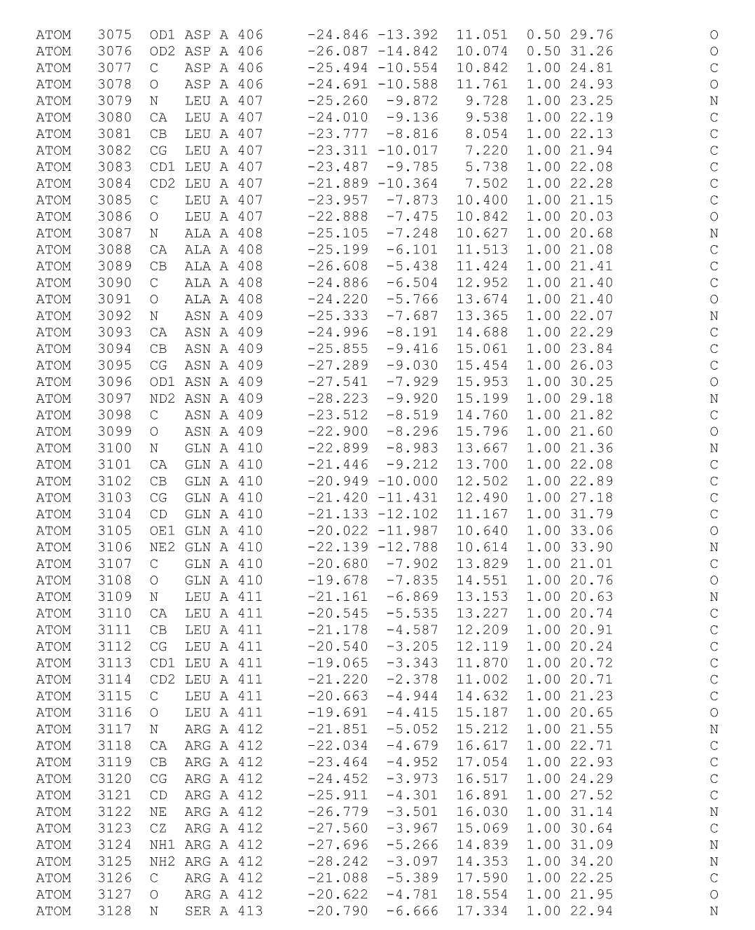

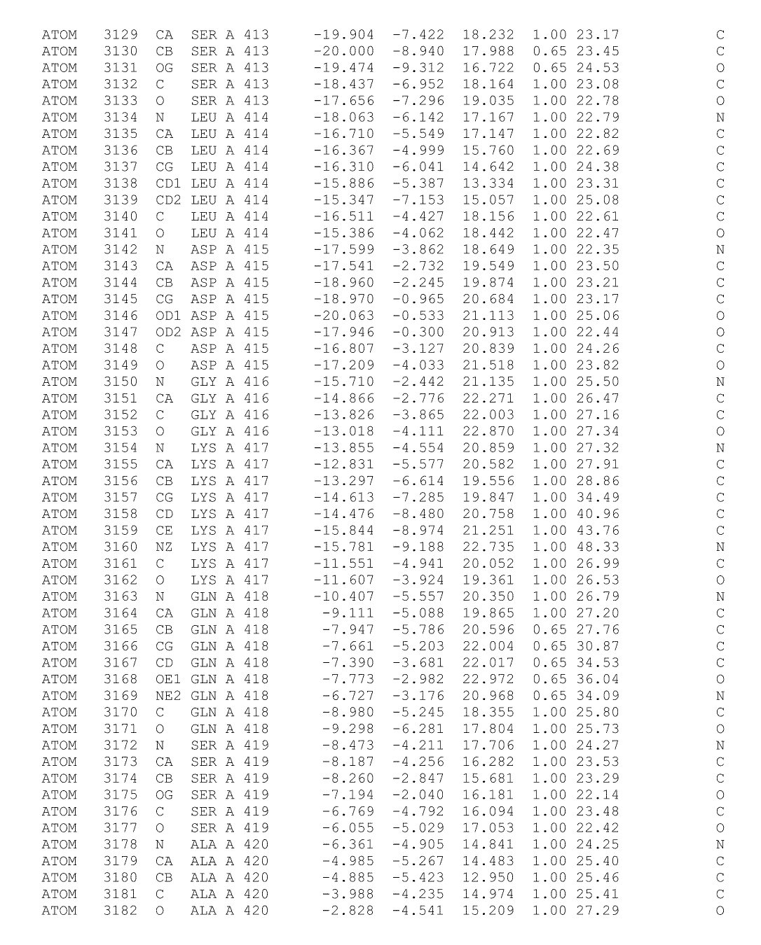

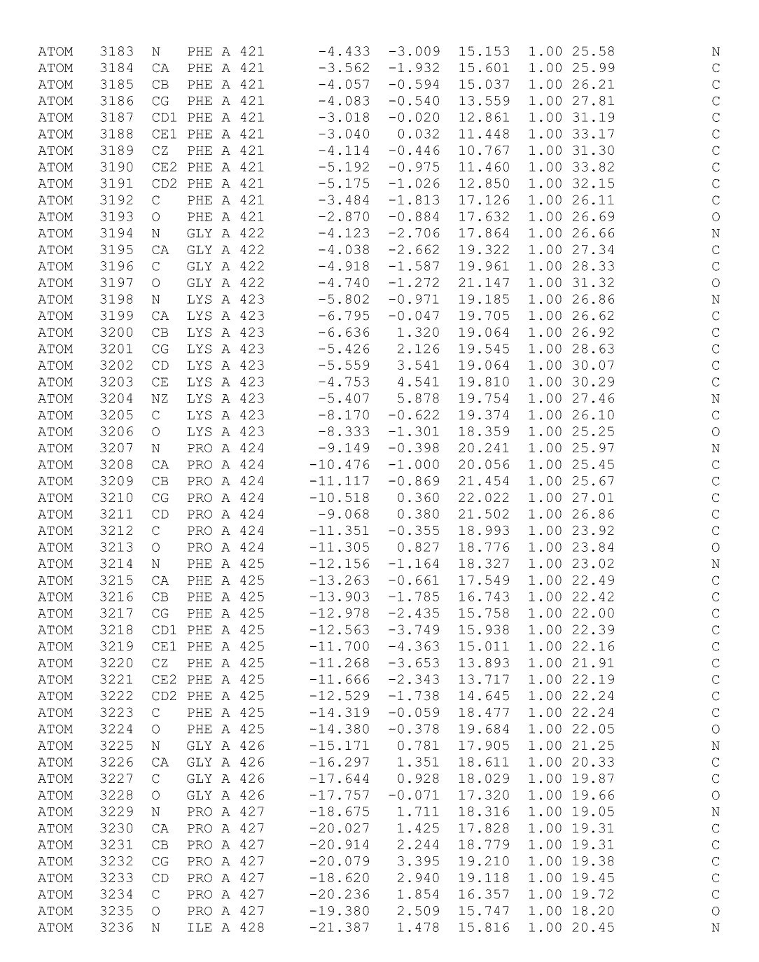

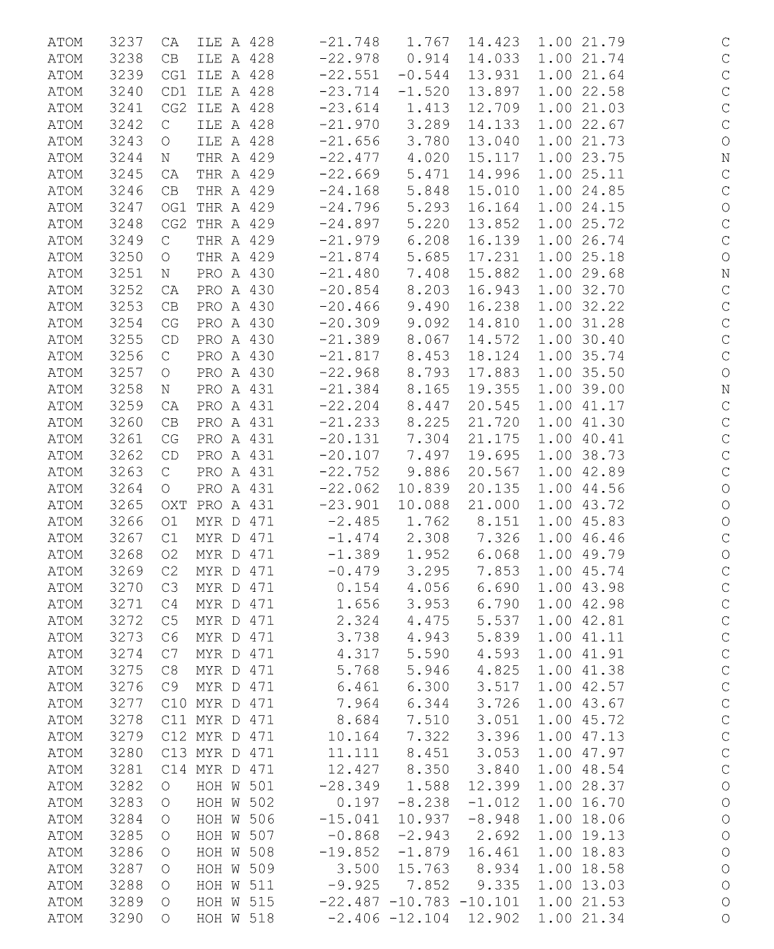

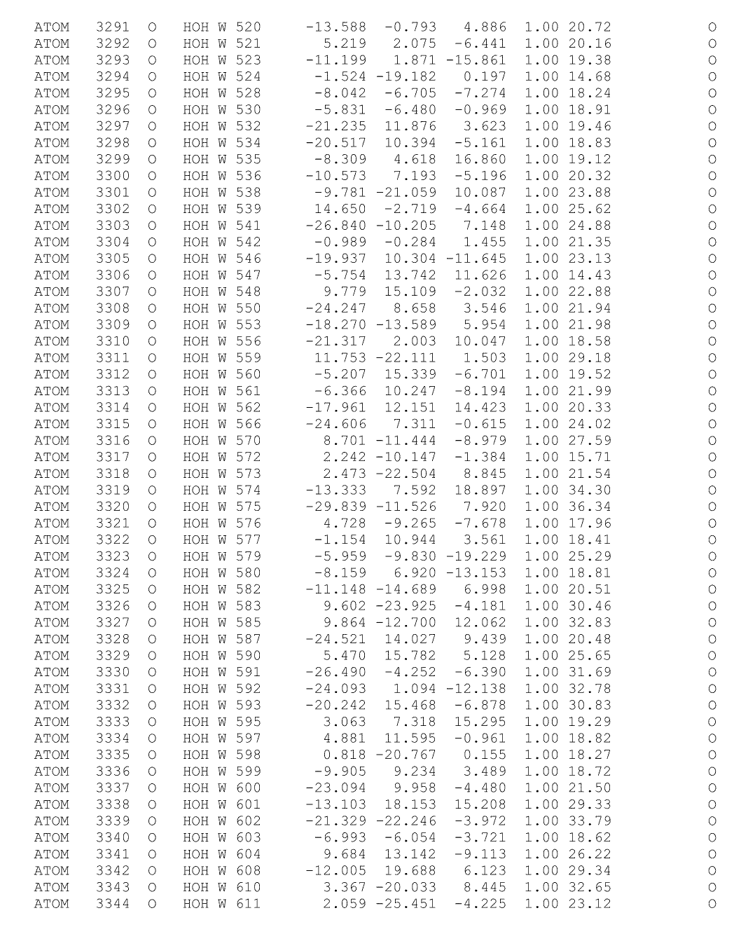

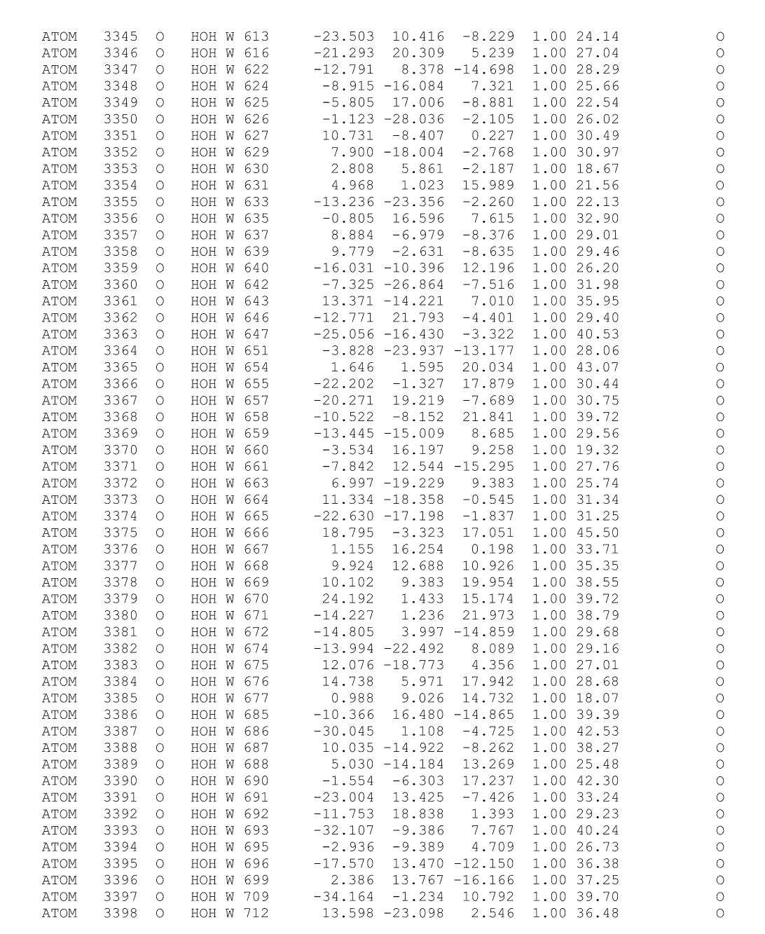

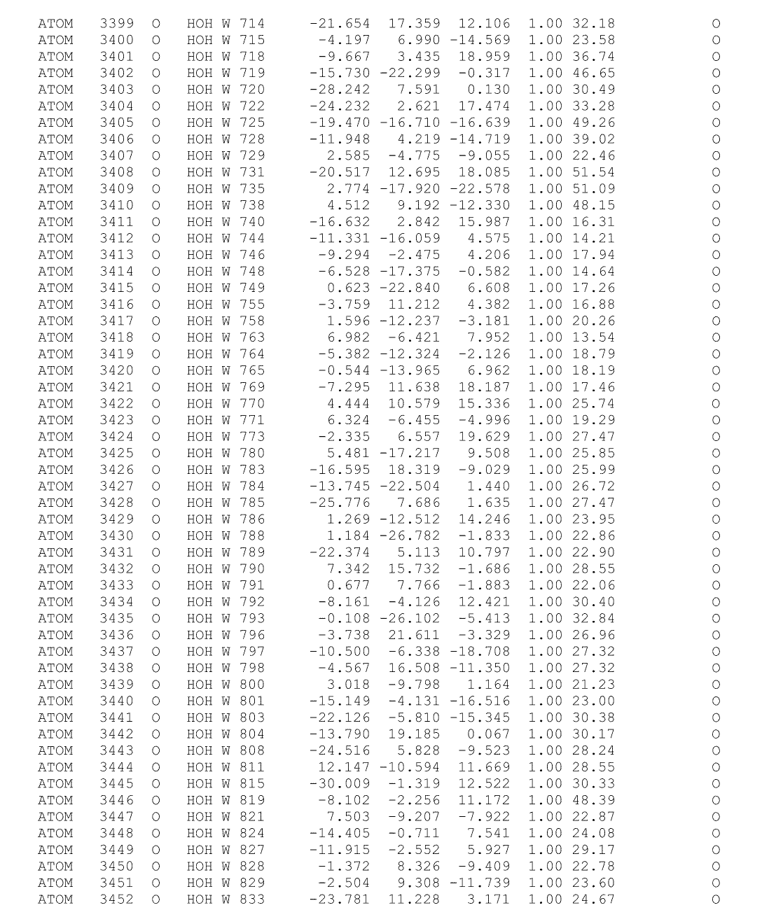

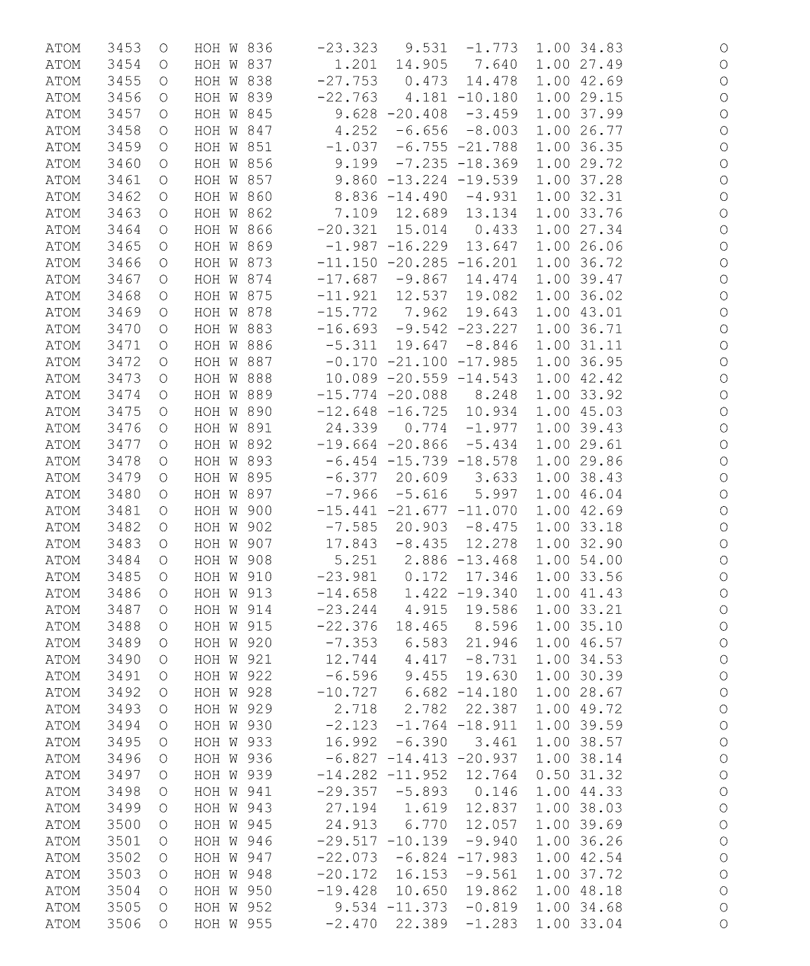

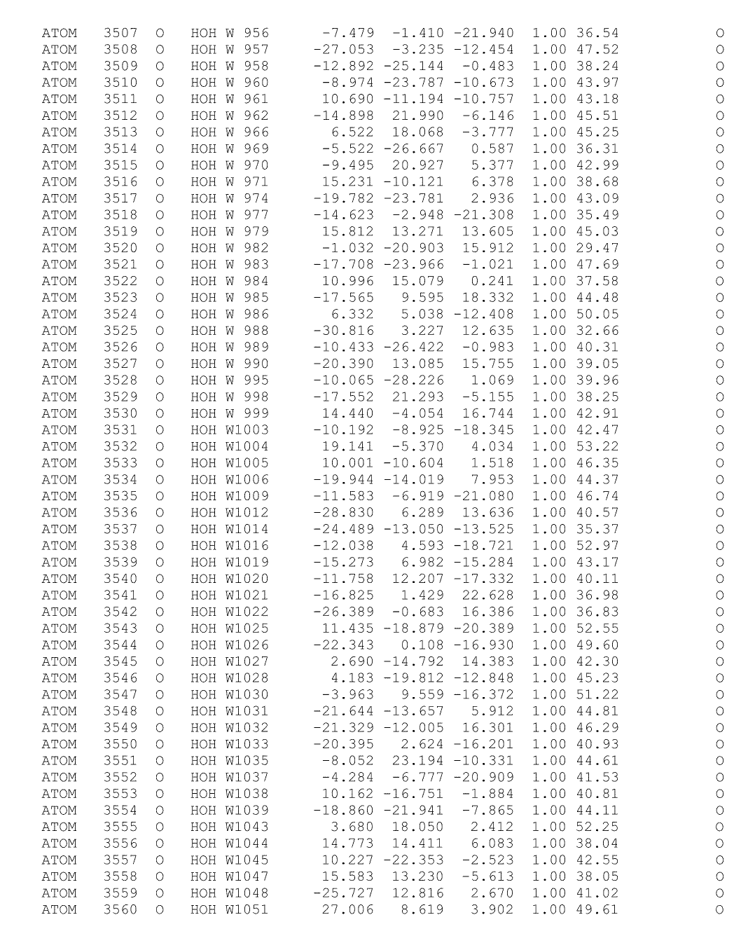

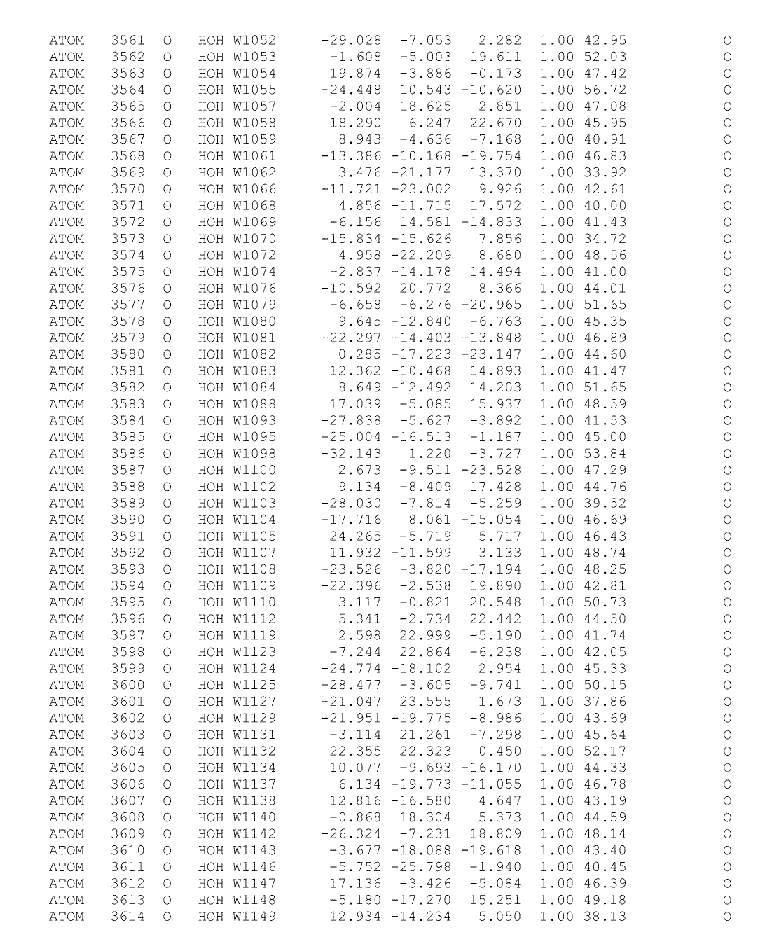

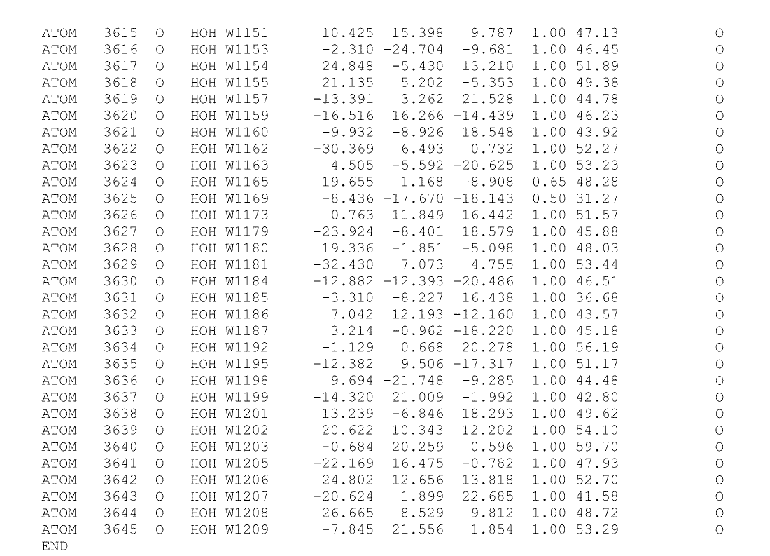

The invention uses a 3D model of the parent polypeptide with a substrate analogue comprising a straight-chain fatty acyl group. FIG. 1 gives the coordinates for a 3D model of C. antarctica lipase A with myristic acid as a substrate analogue.

### Selection of Amino Acid Residue

A carbon atom (number n) in the fatty acid (or other substrate analogue) is selected so as to match the desired -length specificity, e.g. n=3 if a variant is desired with increased selectivity for chain length<=3 relative to chain length>4, and an amino acid residue is selected in the 3D model having a non-hydrogen atom within 10 Å of the selected carbon atom.

In the model in FIG. 1, the following residues have a non-H atom within 10 Å of a carbon atom in the myristic acid: 82-87, 108, 132-133, 138, 140-142, 145, 172-179, 182, 202-216, 220-232, 235, 238, 241-242, 257, 264, 267-268, 275-277, 280, 282-288, 290-296, 298-299, 304, 320, 324-328, 356-357, 360 and 420-421. The selected residue may particularly correspond to any of residues 139, 140, 205, 208, 211, 212, 215, 216, 223, 225, 227, 228, 231, 235, 238, 241, 242, 286, 291, 295 or 326 of SEQ ID NO: 1.

More particularly, any of the following residues may be selected:
For chain length (n) 1-2, residue 139, 205, 211, 215 or 326.
For n=3-4, residue 139, 140, 211, 215 or 223.
For n=5-7, residue 208, 212, 225 or 227.
For n=7-10, residue 209, 231, 238, 2412, 286, 291 or 295.
For n>12, residue 209, 212, 216, 228, 238, 241, 291 or 295.
For n>18, residue 216, 235, 238, 242, 291 or 295.

### Altered Amino Acid Sequence

The selected residue may be substituted with a different residue, particularly with a larger residue. Amino acid residues are ranked as follows from smallest to largest: (an equal sign indicates residues with sizes that are practically indistinguishable):

G<A<S=C<V=T=P<L=I=N=D=M<E=Q<K<H<R=F<Y<W

The substitution may particularly be made with a slightly larger residue, e.g. one or two steps larger in the above ranking.

The substitution may particularly be with another residue of the same type where the type is negative, positive, hydrophobic or hydrophilic. The negative residues are D,E, the positive residues are K,R, the hydrophobic residues are A,C,F,G,I,L,M,P,V,W,Y, and the hydrophilic residues are H,N,Q,S,T.

Alternatively, an amino acid insertion may be made at the N- or C-terminal side of the selected residue, particularly an insertion of 1-2 residues.

### Particular Variants

The variant may particularly comprise one or more of the following substitutions: 1140FYW, P205WYF, T21FW or L231Y.

The variant polypeptide may have the amino acid sequence of SEQ ID NO: 1 with one of the following sets of substitutions:

P205W

P205Y

P205F

T211F

P205F T211W

T211W

P205W T211W

I140F P205F T211W

I140F P205Y

I140W P205F

P205W T211F

I140Y T211F

I140Y P205W

I140Y

I140F P205W T211W

I140W P205W

I140W

I140W P205W T211Y

L231Y

### Nomenclature for Amino Acid Alterations

In this specification, an amino acid substitution is described by use of one-letter codes, e.g. P205W. Multiple substitutions are concatenated, e.g. P205F T211W to indicate a variant with two substitutions. P205WYF is used to indicate alternatives, i.e. substitution of P205 with W, Y or F.

### Use of Lipolytic Enzyme Variant

The variants of the invention have increased selectivity for short-chain fatty acyl groups. They may be used, e.g., in the following applications:
Release of free fatty acids (FFA) for flavor development in food products, e.g. in cheese ripening. M. Hanson, ZFL, 41 (10), 664-666 (1990).
Enzyme modified cheese (EMC) for use as flavoring for various food products including process cheese, dressing and snack.
Short chain fatty acids for antimicrobials. Ricke S C, Poultry Science, Vol 82 (4) pp. 632-639 (2003) April.
Production of short-chain acid terpenyl esters for the food industry. Laboret F et al., Applied Biochemistry and Biotechnology, Vol. 82 (3) pp. 185-198 (1999) December.
Butyrate for anti-cancer effect. Williams E A et al., Proceedings of the Nutrition Society, Vol. 62 (1) pp. 107-115 (2003) February.
Biocatalysis for ester synthesis of hydrolysis. O Kirk et al., Organic Process Research and Development, Vol. 6 (4) pp 446-451 (2002). M. Svedendahl et al., J.AOCS, 2005, 127, 17988-17989. I Gill et al., Bioorganic and Medicinal Chemistry Letters, 16, 3, 2006, pp 705-709.

### EXAMPLES

### Example 1

### Chain-Length Specificity of Lipolytic Enzyme Variants

Two variants of C. antarctica lipase A were tested for their hydrolytic activity on three different triglycerides and compared to the parent enzyme. The two variants have the amino acid sequence of SEQ ID NO: 1 with the substitution P205F and T211F, respectively.

The testing was done by the method described in WO2005040410A1, and the activity is expressed on a scale from A (Best) to E (None).

| | Tripropionin (C3) | Tributyrin (C4) | Olive oil (18:1) |
|---|---|---|---|
| P205F | B | D | D |
| T211F | B | C | E |
| Parent | B | C | A |

The results show that, compared to the parent enzyme, both variants have a higher selectivity for short-chain fatty acyl groups.

1. A method of preparing a polypeptide, comprising
a) providing a three dimensional model of a parent polypeptide having lipase activity and at least 80% identity to SEQ ID NO: 1 and a substrate analogue comprising a straight-chain fatty acyl group,
b) selecting a chain length (n) and identifying the corresponding carbon atom in the fatty acyl group,
c) selecting an amino acid residue in SEQ ID NO: 1 which has a non-hydrogen atom within 10 Å of the selected carbon atom in the model,
d) providing an altered amino acid sequence which is at least 80% identical to SEQ ID NO: 1, and wherein the difference from SEQ ID NO: 1 comprises substitution of the selected residue or insertion of at least one residue adjacent to the selected residue,
e) preparing an altered polypeptide having the altered amino acid sequence,
f) determining the hydrolytic activity of the altered polypeptide on fatty acyl ester bonds in two substrates having fatty acyl groups with different length, and
g) selecting an altered polypeptide which has an altered chain-length specificity compared to the polypeptide of SEQ ID NO: 1.

2. The method of claim 1 wherein the selected residue has a non-hydrogen atom within 5 Å of the selected carbon atom.

3. The method of claim 1 wherein the selected residue corresponds to any of residues 82-87, 108, 132-133, 138-142, 145, 172-179, 182, 202-216, 220-232, 235, 238, 241-242, 257, 264, 267-268, 275-277, 280, 282-288, 290-296, 298-299, 304, 320, 324-328, 356-357, 360 and 420-421 of SEQ ID NO: 1.

4. The method of claim 1 wherein the selected residue corresponds to residue 139, 140, 205, 208, 211, 212, 215, 216, 223, 225, 227, 228, 231, 235, 238, 241, 242, 286, 291, 295 or 326 of SEQ ID NO: 1.

5. The method of claim 1 wherein n is 1 or 2, and the selected residue corresponds to residue 139, 205, 211, 215 or 326 of SEQ ID NO: 1.

6. The method of claim 1 wherein n is 3 or 4, and the selected residue corresponds to residue 139, 140, 211, 215 or 223 of SEQ ID NO: 1.

7. The method of claim 1 wherein n is in the range 5-7, and the selected residue corresponds to residue 208, 212, 225 or 227 of SEQ ID NO: 1.

8. The method of claim 1 wherein n is in the range 7-10, and the selected residue corresponds to residue 209, 231, 238, 241, 286, 291 or 295 of SEQ ID NO: 1.

9. The method of claim 1 wherein n is in the range 12-17, and the selected residue corresponds to residue 209, 212, 216, 228, 238, 241, 291 or 295 of SEQ ID NO: 1.

10. The method of claim 1 wherein n is 18 or larger, and the selected residue corresponds to residue 216, 235, 238, 242, 291 or 295 of SEQ ID NO: 1.

11. The method of a claim 1 wherein the altered chain-length specificity is a lower ratio of activity towards fatty acyl ester bonds in a first and a second substrate wherein the first fatty acid group has more than n carbon atoms and the second fatty acyl group has n or fewer carbon atoms.

12. The method of claim 1 wherein the two substrates are triglycerides.

13. The method of claim 1 wherein the substitution is made with a larger residue.

14.-15. (canceled)

16. A polypeptide which:
a) has lipolytic enzyme activity, and
b) has an amino acid sequence which has at least 80% identity to SEQ ID NO: 1 and has a different residue at a position or an insertion adjacent to a residue corresponding to any of residues 82-87, 108, 132-133, 138, 140-142, 145, 172-179, 182, 202-216, 220-232, 235, 238, 241-242, 257, 264, 267-268, 275-277, 280, 282-288, 290-296, 298-299, 304, 320, 324-328, 356-357, 360 and 420-421 of SEQ ID NO: 1.

17. The polypeptide of claim 16 wherein the selected residue corresponds to any of residues 139, 140, 205, 208, 211, 212, 215, 216, 223, 225, 227, 228, 231, 235, 238, 241, 242, 286, 291, 295 or 326 of SEQ ID NO: 1.

18. The polypeptide of claim 16 wherein the different residue is a larger residue.

19. The polypeptide of claim 16 wherein the different residue is another residue of the same type where the type is negative, positive, hydrophobic or hydrophilic.

20. The polypeptide of claim 16 wherein the insertion consists of one or two residues inserted at the N- or C-side of the selected residue.

21. The polypeptide of claim 16 wherein the difference from SEQ ID NO: 1 comprises a substitution corresponding to I140FYW, P205WYF, T21FW or L231Y.

22. The polypeptide of claim 16 which has an amino acid sequence differing from SEQ ID NO: 1 as follows: P205W, P205Y, P205F, T211F, P205F T211W, T211W, P205W T211W, I140F P205F T211W, I140F P205Y, I140W P205F, P205W T211F, I140Y T211F, I140Y P205W, I140Y, I140F P205W T211W, I140W P205W, I140W, I140W P205W T211Y, L231Y.
